(19) United States
(12) Patent Application Publication
(10) Pub. No.: US 2024/0299347 A1
(43) Pub. Date: Sep. 12, 2024

(54) METHODS AND COMPOSITIONS TO TREAT HUNTINGTON'S DISEASE BY TARGETING ALOX5- MEDIATED FERROPTOSIS

(71) Applicant: The Trustees of Columbia University in the City of New York, New York, NY (US)

(72) Inventor: Wei GU, Paramus, NJ (US)

(21) Appl. No.: 18/601,908

(22) Filed: Mar. 11, 2024

Related U.S. Application Data
(60) Provisional application No. 63/489,732, filed on Mar. 10, 2023.

(51) Int. Cl. A61K 31/381; A61K 31/122; A61K 31/19; A61K 31/352; A61K 31/405; A61K 31/4155; A61K 31/4178; A61K 31/4245; A61K 31/7088; A61K 38/46; A61P 25/28; C12N 15/113; G01N 33/68

(52) U.S. Cl. CPC A61K 31/381; A61K 31/122; A61K 31/19; A61K 31/352; A61K 31/405; A61K 31/4155; A61K 31/4178; A61K 31/4245; A61K 31/7088; A61K 38/465; A61P 25/28; C12N 15/1137; C12Y 113/11034; G01N 33/6896; C12N 2310/14; G01N 2333/90241; G01N 2800/2835

(57) ABSTRACT

Disclosed herein are compositions and methods for treating or preventing Huntington's disease. In one aspect, the disclosed methods relate to targeting arachidonate 5-lipoxygenase (ALOX5) and 5-lipoxygenase-activating protein (FLAP). The compositions and methods disclosed herein can be used as disease modifying therapies to enable treatment of Huntington's disease and related disorders earlier in disease progression and improve clinical outcomes.

This application claims the benefit of and priority to U.S. Application No. 63/489,732, filed Mar. 10, 2023, entitled “ALOX5-MEDIATED FERROPTOSIS ACTS AS A DISTINCT CELL DEATH PATHWAY UPON OXIDATIVE STRESS IN HUNTINGTON'S DISEASE” the contents of which is hereby incorporated by reference in its entirety.

### STATEMENT REGARDING FEDERALLY SPONSORED RESEARCH OR DEVELOPMENT

This invention was made with government support under CA253059 awarded by the National Institutes of Health. The government has certain rights in the invention.

All patents, patent applications and publications, and other literature references cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Huntington's disease (HD) is an autosomal dominant hereditary neurodegenerative disease characterized by progressive cognitive, behavioral, motor dysfunctions and short life-spans. HD is caused by a CAG trinucleotide repeat expansion in Exon 1 of the HTT gene, which results in an expanded polyglutamine (polyQ) tract in the encoded huntingtin protein, referred to as mutant huntingtin (mHTT). The mHTT exhibits toxic gain of functions causing neuronal dysfunction and cell death. The proteolytic cleavage of mHTT is a key event in the molecular pathogenesis of HD and it is believed that the N-terminal fragment containing the expanded polyglutamine of mHTT plays an important role in the pathogenesis of HD. Although the genetic cause of HD is well established, the cellular and molecular mechanisms involved in mHTT-mediated early neuronal dysfunction and late neurodegeneration are not fully understood.

### SUMMARY

In certain aspects, described herein is a method of treating or preventing Huntington's disease in a subject in need thereof, comprising administering to said subject a composition that targets the acyl-CoA synthetase long-chain family member 4 (ACSL4)-independent ferroptosis pathway.

In certain aspects, described herein is a method of treating or preventing Huntington's disease in a subject in need thereof, comprising: (i) identifying the subject as expressing of mutant huntingtin (mHTT), and (ii) administering to said subject a composition that targets the acyl-CoA synthetase long-chain family member 4 (ACSL4)-independent ferroptosis pathway.

In certain aspects, described herein is a method of treating or preventing Huntington's disease in a subject in need thereof, comprising: (i) identifying the subject as having an increased expression level of arachidonate 5-lipoxygenase (ALOX5), and (ii) administering to said subject a composition that targets the acyl-CoA synthetase long-chain family member 4 (ACSL4)-independent ferroptosis pathway.

In certain aspects, described herein is a method of treating or preventing Huntington's disease in a subject in need thereof, comprising: (i) identifying the subject as having an increased expression level of 5-lipoxygenase-activating protein (FLAP), and (ii) administering to said subject a composition that targets the acyl-CoA synthetase long-chain family member 4 (ACSL4)-independent ferroptosis pathway.

In some embodiments, the subject expresses mHTT, an increased level of ALOX5, an increased level of FLAP, or a combination thereof. In some embodiments, expression of mHTT is determined from a sample from the subject. In some embodiments, expression level of ALOX5 or FLAP is determined from a sample from the subject. In some embodiments, expression or expression level is a protein level, a mRNA expression level, or combination thereof.

In some embodiments, the subject further expresses increased level of ALOX5, an increased level of FLAP, or a combination thereof. In some embodiments, the subject further expresses mHTT, an increased level of FLAP, or a combination thereof. In some embodiments, the subject further expresses mHTT, or an increased level of ALOX5 or a combination thereof.

In some embodiments, the composition reduces ALOX5 expression in the subject compared to ALOX5 expression in a subject suffering from Huntington's disease or compared to ALOX5 expression in the subject before administration of the composition. In some embodiments, the composition reduces FLAP expression in the subject compared to FLAP expression in a subject suffering from Huntington's disease or compared to FLAP expression before administration of the composition. In some embodiments, the composition inhibits or reduces ALOX5 expression in the subject. In some embodiments, the composition inhibits or reduces FLAP expression in the subject. In some embodiments, the composition comprises Zileuton. In some embodiments, the composition comprises MK.886. In some embodiments, the composition comprises docebenone (AA 861). In some embodiments, the composition comprises boswellic acids. Insome embodiments, the composition comprises atreleuton (ABT-761 or VIA-2291). In some embodiments, the composition comprises setileuton (1,3,4-oxadiazole MK-0633). In some embodiments, the composition comprises PF-4191834 or CJ-13610. In some embodiments, the composition comprises Flavocoxid.

In some embodiments, the composition comprises a ALOX5 small interfering ribonucleic acid (siRNA). In some embodiments, the composition comprises a FLAP small interfering ribonucleic acid (siRNA).

In some embodiments, the composition comprises an ALOX5 short-hairpin ribonucleic acid (shALOX5). In some embodiments, the composition comprises a FLAP short-hairpin ribonucleic acid (shFLAP).

In some embodiments, the composition comprises a guide RNA or a single-molecule guide RNA comprising a spacer sequence that is complementary to a portion of a nucleic acid sequence encoding ALOX5. In some embodiments, the composition comprises a guide RNA or a single-molecule guide RNA comprising a spacer sequence that is complementary to a portion of a nucleic acid sequence encoding FLAP. In some embodiments, the guide RNA or the single molecule guide RNA is pre-complexed with a DNA endonuclease. In some embodiments, the DNA endonuclease is a Cas9 or dCas9 endonuclease.

In some embodiments, the composition comprises a vector. In some embodiments, the composition comprises a viral vector, wherein the viral vector encapsulates a nucleic acid encoding the shALOX5, a nucleic acid encoding the shFLAP, a nucleic acid encoding the guide ribonucleic acid or a single molecule guide RNA comprising a spacer sequence that is complementary to a portion of a nucleic acid sequence encoding ALOX5, or a nucleic acid encoding the guide ribonucleic acid or a single molecule guide RNA comprising a spacer sequence that is complementary to a portion of a nucleic acid sequence encoding FLAP. In some embodiments, the viral vector is an adeno-associated vector (AAV).

In some embodiments, the subject is a mammal. In some embodiments, the mammal is a human. In some embodiments, the composition is delivered systemically.

In some embodiments, the human subject has an increased expression level of ALOX5 compared to a subject not suffering from Huntington's disease. In some embodiments, the human subject has an increased expression level of FLAP compared to a subject not suffering from Huntington's disease.

In certain aspects, described herein is a composition for treating or preventing Huntington's disease, comprising a composition targeting the ACSL4-independent ferroptosis pathway. In some embodiments, the composition reduces arachidonate 5-lipoxygenase (ALOX5) expression in a subject in need thereof. In some embodiments, the composition reduces 5-lipoxygenase-activating protein (FLAP) expression in a subject in need thereof.

In some embodiments, the composition comprises Zileuton. In some embodiments, the composition comprises MK.886. In some embodiments, the composition comprises docebenone (AA 861). In some embodiments, the composition comprises boswellic acids. Insome embodiments, the composition comprises atreleuton (ABT-761 or VIA-2291). In some embodiments, the composition comprises setileuton (1,3,4-oxadiazole MK-0633). In some embodiments, the composition comprises PF-4191834 or CJ-13610. In some embodiments, the composition comprises Flavocoxid. In some embodiments, the composition comprises an ALOX5 small interfering ribonucleic acid (siALOX5). In some embodiments, the composition comprises a FLAP small interfering ribonucleic acid (siFLAP). In some embodiments, the composition comprises an ALOX5 short hairpin ribonucleic acid (shALOX5). In some embodiments, the composition comprises a FLAP short hairpin ribonucleic acid (shFLAP). In some embodiments, the composition comprises a guide ribonucleic acid or a single molecule guide RNA comprising a spacer sequence that is complementary to a portion of a nucleic acid sequence encoding ALOX5. In some embodiments, the composition comprises a guide ribonucleic acid or a single molecule guide RNA comprising a spacer sequence that is complementary to a portion of a nucleic acid sequence encoding FLAP. In some embodiments, the guide RNA or the single molecule guide RNA is pre-complexed with a DNA endonuclease. In some embodiments, the DNA endonuclease is a Cas9 or dCas9 endonuclease.

In some embodiments, the composition further comprises a viral vector, wherein the viral vector encapsulates a nucleic acid encoding the shALOX5, the shFLAP, the guide ribonucleic acid or a single molecule guide RNA comprising a spacer sequence that is complementary to a portion of a nucleic acid sequence encoding ALOX5, or the guide ribonucleic acid or a single molecule guide RNA comprising a spacer sequence that is complementary to a portion of a nucleic acid sequence encoding FLAP. In some embodiments, the viral vector is an AAV vector.

In certain aspects, described herein is small interfering ribonucleic acid (siRNA) targeting the acyl-CoA synthetase long-chain family member 4 (ACSL4)-independent ferroptosis pathway. In some embodiments, the siRNA is an ALOX5 small interfering ribonucleic acid (siALOX5). In some embodiments, the siRNA is a FLAP small interfering ribonucleic acid (siFLAP). In certain aspects, described herein is a vector comprising a SiRNA.

In certain aspects, described herein is a nucleic acid comprising a sequence encoding a short hairpin ribonucleic acid (shRNA) targeting the acyl-CoA synthetase long-chain family member 4 (ACSL4)-independent ferroptosis pathway. In certain aspects, described herein is a vector comprising any of the nucleic acid described above. In certain aspects, described herein is a viral vector comprising the nucleic acid described above. In some embodiments, the viral vector is an AAV vector.

In certain aspects, described herein is a method of diagnosing Huntington's disease in a subject comprising: (i) determining the presence of mutant huntingtin (mHTT) in the subject. In certain aspects, described herein is a method of diagnosing Huntington's disease in a subject comprising: (i) determining the level of arachidonate 5-lipoxygenase (ALOX5) in the subject; and (ii) diagnosing the subject with Huntington's disease if the level of ALOX5 expression in the subject is increased as compared to a subject not suffering from Huntington's disease. In some embodiments, the method further comprises determining the level of FLAP in the subject. In some embodiments, the method further comprises determining the whether the subject expresses mHTT. In certain aspects, described herein is a method of diagnosing Huntington's disease in a subject comprising: (i) determining the level of 5-lipoxygenase-activating protein (FLAP) in the subject; and (ii) diagnosing the subject with Huntington's disease if the level of FLAP expression in the subject is increased as compared to a subject not suffering from Huntington's disease. In some embodiments, the method further comprises determining the level of ALOX5 in the subject. In some embodiments, the method further comprises determining whether the subject expresses mHTT. In some embodiments the level of ALOX5, FLAP, and/or mHTT is determined from a sample from the subject. In some embodiments, the subject is diagnosed with Huntington's disease if the level of both FLAP and ALOX5 expression in the subject is increased as compared to a subject not suffering from Huntington's disease. In some embodiments, the subject is diagnosed with Huntington's disease if the level of FLAP expression in the subject is increased as compared to a subject not suffering from Huntington's disease and the subject expresses mHTT. In some embodiments, the subject is diagnosed with Huntington's disease if the level of ALOX5 expression in the subject is increased as compared to a subject not suffering from Huntington's disease and the subject expresses mHTT. In some embodiments, the subject is diagnosed with Huntington's disease if the level of both ALOX5 and FLAP expression in the subject is increased as compared to a subject not suffering from Huntington's disease and the subject expresses mHTT. In some embodiments, the subject is a mammal. In some embodiments, the mammal is a human.

### BRIEF DESCRIPTION OF THE DRAWINGS

The patent or application file contains at least one drawing executed in color.

FIGS. 1A-E. HTTQ94 expression sensitizes neuronal cells and other cell types to ferroptosis. (A) Western blot analysis for HTTQ94 from HTTQ94 Tet-on HT-22 cells treated with doxycycline (0.5 μg/ml) for 2, 4, 8 and 16 h. (B) HTTQ94 Tet-on HT-22 cells were pre-incubated with doxycycline (0.5 μg/ml) for 4 h, and then treated with Erastin (1 μM) for 12 h with/without Ferr-1 (2 μM). (C) Representative phase-contrast images of cell death, related to panel B. (D) Western blot analysis for mutant HTT fragment (HTTQ94) and normal HTT fragment (HTTQ19) expression in the Tet-on H1299 cells treated with doxycycline (0.5 μg/ml; Tet) for 16 h. (E) Cell death assay. Control, HTTQ94 and HTTQ19 fragment Tet-on H1299 cells pre-incubated with doxycycline (0.5 μg/ml) for 16 h were treated with Erastin (30 μM) for 48 h with/without Ferr-1 (2 μM). Cell death were calculated from three replicates; Data shown in (B) and (E) are the means±SD. P-values were derived from two-tailed unpaired t-test. (***) P≤0.001, (**) P≤0.01, (*) P≤0.05, (n.s.) P >0.05.

FIGS. 2A-F. The role of ACSL4 in mHTT-induced ferroptosis and the life span of the HD mice. (A) Cell death assay. HTTQ94 Tet-on HT-22 cells pre-incubated with doxycycline (0.5 μg/ml) for 4 h were treated with Erastin (1 μM) for 12 h in the presence or absence of ferrostatin-1 (Ferr-1, 2 μM) or ACSL4 inhibitors (rosiglitazone, ROSI, 10 μM; troglitazone, TRO, 10 μM). (B) Cell death assay. HTTQ94 Tet-on SK-N-BE(2)C cells pre-incubated with doxycycline (0.5 μg/ml) for 16 h were treated with Erastin (40 μM) for 32 h in the presence or absence of ferrostatin-1 (Ferr-1, 2 μM), rosiglitazone (ROSI, 10 μM) and troglitazone(TRO, 10 μM). (C) Western blot analysis for ACSL4 and HTTQ94 in HTTQ94 Tet-on SK-N-BE(2)C control and ACSL4 crispr cells treated with doxycycline (0.5 μg/ml) for 16 h. (D) Cell death assay. HTTQ94 tet-on SK-N-BE(2)C control crispr and ACSLA crispr cells were pre-incubated with doxycycline (0.5 μg/ml) for 16 h, and then treated with erastin (40 μM) for 32 h with/without Ferr-1 (2 μM). (E) Western blot analysis for ACSL4 from the cerebral cortex tissues of HD and HD/Acs14null mice. (F) Kaplan-Meier survival curves of HD (n=21 independent mice); HD/Acs14null (n=7 independent mice) mice. Cell death were calculated from three replicates. Data shown in (A), (B), and (D) are the means±SD. P-values were derived from two-tailed unpaired t-test. (***) P<0.001. In (F), P value (HD versus HD/acsl4null) was calculated using log-rank Mantel-Cox test. P=0.1356.

FIGS. 3A-E. HTTQ94 is able to induce the ACSL4-independent ferroptosis upon ROS stress. (A) Representative phase-contrast images of cell death from the HTTQ94 tet-on HT-22 cells. HTTQ94 Tet-on HT-22 cells were pre-incubated with doxycycline (0.5 μg/ml) for 4 h, and then treated with TBH (10 μM) for 8 h in the presence or absence of the ferroptosis inhibitors (ferrostatin-1, Ferr1, 2 μM; liproxstatin-1, Lipor1, 2 μM and DFO, 100 μM), apoptosis inhibitor (Z-VADFMK, zVAD, 10 μM), autophagy inhibitor (3-methylademine, 3 MA, 2 mM) or necroptosis inhibitor (necrostatin-1, Nec1, 10 μM). (B) Quantification of cell death, related to panel A. Three replicates for each group. (C) HTTQ94 tet-on SK-N-BE(2)C control crispr and ACSLA crispr cells were pre-incubated with doxycycline (0.5 μg/ml) for 16 h, and then treated with TBH (350 μM) for 24 h with/without Ferr-1 (2 μM). (D) Western blot analysis of GPX4, ACSL4 and HTTQ94 in HTTQ94 Tet-on SK-N-BE(2)C control crispr and GPX4/ACSL4 double crispr cells treated with doxycycline (0.5 μg/ml) for 16 h. (E) HTTQ94 tet-on SK-N-BE(2)C control crispr and GPX4/ACSL4 double crispr cells were pre-incubated with doxycycline (0.5 μg/ml) for 16 h, and then treated with TBH (350 μM) for 24 h with/without Ferr-1 (2 μM). Cell death were calculated from three replicates; Data shown in (B), (C), and (E) are means±SD. P-values were derived from two-tailed unpaired t-test. (***) P≤0.001, (**) P≤0.01, (*) P≤0.05, (n.s.) P >0.05.

FIGS. 4A-G. ALOX5 is required for HTTQ94 mediated ferroptosis by ROS-induced stress and glutamate. (A) Cell death assay in HTTQ94 Tet-on SK-N-BE(2)C cells with different ALOXs knockdown. Cells were transfected with control siRNA (ctrl) or ALOX family specific siRNAs, followed by pre-incubation with doxycycline (0.5 μg/ml) for 16 h, then treated with TBH (350 μM) for 24 h. (B) Q-PCR analysis of the knockdown efficiency of ALOX family members in HTTQ94 Tet-on SK-N-BE(2)C cells transfected with control siRNA or ALOX family specific siRNAs. (C) Cell death assay in HTTQ94 Tet-on HT-22 cells with ALOX5 knockdown. Cells were transfected with control siRNA (ctrl) or ALOX5 specific siRNA, and then pre-incubated with doxycycline (0.5 μg/ml) for 4 h, followed by TBH (10 μM) treatment for 8 h. left panel: ALOX5 knockdown efficiency; right panel: cell death assay. (D) Western blot analysis of ALOX5 and HTTQ94 in HTTQ94 Tet-on SK-N-BE(2)C control crispr and ALOX5 crispr cells treated with doxycycline (0.5 μg/ml) for 16 h. (E) Cell death assay for mHTT tet-on SK-N-BE(2)C control crispr and ALOX5 crispr cells. Cells were pre-incubated with doxycycline (0.5 μg/ml) for 16 h, followed by incubation with TBH (350 μM) for 24 h with/without Ferr-1 (2 μM). (F) FACS analysis of Lipid ROS production in HTTQ94 Tet-on SK-N-BE(2)C control crispr and ALOX5 crispr cells. Cells were pre-incubated with doxycycline (0.5 μg/ml) for 16 h, and then treated with TBH (350 μM) for 6 h. Lipid ROS was stained with C11-BODIPY. (G) Cell death assay in HTTQ94 Tet-on HT-22 cells. Cells were transfected with control siRNA (ctrl) or ALOX5 siRNA, followed by incubation with doxycycline (0.5 μg/ml) and glutamate (10 mM) for 16 h in the presence or absence of Ferr-1 (2 μM). Cell death were calculated from three replicates; Data shown in (A), (C), (E) and (G) are means±SD. P-values were derived from two-tailed unpaired t-test. (***) P≤0.001.

FIGS. 5A-G. Mechanistic insight into HTTQ94-induced ALOX5 activation. (A) Western blot analysis of FLAP and HTTQ94 or HTTQ19 in HEK293 cells transfected with an HA-FLAP expressing plasmid and either an empty vector, HTTQ94-GFP or HTTQ19-GFP expressing vector. (B) Western blot analysis of endogenous FLAP, ALOX5 in HTTQ94 Tet-on SK-N-BE(2)C cells incubated with doxycycline (0.5 μg/ml) for 4 h, 8 h and 16 h. (C) Densitometry quantification of FLAP protein levels calculated using ImageJ software and plotted for half-life determination corresponding to FIG. 11. (D) Co-IP of GFP-tagged HTTQ94/HTTQ19 and Flag-tagged FLAP in HEK293 cells. Cell lysates were IP with anti-Flag-coupled beads (M2), followed by Western analysis of HTTQ94, HTTQ19 and FLAP. (E) Ubiquitination analysis of FLAP in the presence of HTTQ94 or HTTQ19. HEK293 cells were co-transfected with Flag-FLAG and HA-Ubiquitin (Ub) in the presence of HTTQ94 or HTTQ19. Cell lysates were IP with anti-Flag-coupled beads (M2), followed by Western blot analysis of HA, FLAP, and HTT proteins. (F) Cell death assay for HTTQ94 Tet-on SK-N-BE(2)C cells with FLAP knockdown. Cells were transfected with control siRNA (ctrl) or FLAP specific siRNA, and then pre-incubated with doxycycline (0.5 μg/ml) for 16 h, followed by TBH (350 μM) treatment for 24 h. (G) Cell death assay. HTTQ94 Tet-on HT-22 cells pre-incubated with doxycycline (0.5 μg/ml) for 4 h were treated with TBH (10 μM) for 8 h in the presence or absence of Ferr-1(2 μM), Zileuton (10 μM) or MK886 (10 μM). Cell death were calculated from three replicates; Data shown in (F) and (G) are the means±SD. P-values were derived from two-tailed unpaired t-test. (***) P≤0.001.

FIGS. 6A-H. Loss of ALOX5 ameliorates the phenotypes and significantly extends the life spans of HD mice. (A) Western blot analysis for ALOX5 expression in HD and HD/Alox5null mice brain. (B) Representative images of limb clasping from the HD and HD/Alox5null mice. (C) Open field test.13-weeks-old mice were subjected to open field test, and the distance moved was calculated. Six mice for each group (HD vs HD/Alox5null, p<0.01). (D) Kaplan-Meier survival curves of HD (n=21 independent mice) and HD/Alox5null (n=25 independent mice) mice. P value was calculated using log-rank Mantel-Cox test. (HD vs HD/Alox5null, p<0.0001). (E and F) TfR1 staining on mouse brain slides. WT, HD-N171-82Q, and HD-N171-82Q/Alox5null mice brain paraffin slides were dewaxed, and antigens were retrieved by PH6.0 citric acid solution, followed by incubation with TfR1 antibody. (E) Representative images of TfR1 staining (Four mice for each group). Nuclei were stained with DAPI. (F) Quantification of TfR1 positive striatal neurons. Data are represented as mean±s.e.m. (G) Representative images of TfR1 and DARPP-32 double staining on mouse brain paraffin slides. DARPP-32 was used as a marker for striatal medium spiny neurons (Naia et al, Bio-protocol. 2018). (H) Quantification of TfR1 positive striatal neurons, related to panel G. Data shown in (C), (F), and (H) are the means±SEM. n=6. P-values were derived from two-tailed unpaired t-test. (***) P≤0.001, (**) P≤0.01.

FIGS. 7A-E. The effect of HTTQ94 on ACSL4-dependent ferroptotic responses. (A) Western blot analysis of HTTQ94 in HTTQ94 Tet-on SK-N-BE(2)C cells treated with doxycycline (0.5 μg/ml; Tet) for 2, 4, 8 and 16 h. (B) HTTQ94 Tet-on SK-N-BE(2)C cells pre-incubated with doxycycline (0.5 μg/ml) for 16 h were treated with Erastin (40 μM) for 32 h with/without Ferr-1 (2 μM). (C) Representative phase-contrast images of cell death from, related to panel B. (D) Western blot analysis for ACSL4 in different ACSL4 crispr knockout clones from the HTTQ94 Tet-on SK-N-BE(2)C cell line. (E) Cell death assay. The HTTQ94 Tet-on SK-N-BE(2)C control crispr and four independent ACSL4 crispr cell clones pre-incubated with doxycycline (0.5 μg/ml) for 16 h were treated with erastin (40 μM) for 32 h with/without Ferr-1 (2 μM). Cell death were calculated from three replicates; Data shown in (B) and (E) are means±SD. P-values were derived from two-tailed unpaired t-test. (***) P≤0.001,(**) P≤0.01,(*)P<0.05,(n.s.) P>0.05.

FIGS. 8A-C. HTTQ94 mediated ferroptotic responses upon ROS stress. (A) Representative phase-contrast images of cell death from the HTTQ94 tet-on SK-N-BE(2)C cells. HTTQ94 Tet-on SK-N-BE(2)C cells pre-incubated with doxycycline (0.5 μg/ml) for 16 h were treated with TBH (350 μM) for 24 h in the presence or absence of the ferroptosis inhibitors (ferrostatin-1, Ferr1, 2 μM; liproxstatin-1, Lipor1, 2 μM and DFO, 100 μM), apoptosis inhibitor (Z-VADFMK, zVAD, 10 μM), autophagy inhibitor (3-methylademine, 3 MA, 2 mM) or necroptosis inhibitor (necrostatin-1, Nec1, 10 μM). (B) Quantification of cell death, related to panel A; (C) Cell death assay. Control, HTTQ94 and HTTQ19 fragment Tet-on H1299 cells pre-incubated with doxycycline (0.5 μg/ml) for 16 h were treated with TBH (50 μM) for 24 h with/without Ferr-1 (2 μM). Cell death were calculated from three replicates; Data shown in (B) and (C) are means±SD. P-values were derived from two-tailed unpaired t-test. (***) P≤0.001, (**) P≤0.01, (*) P≤0.05, (n.s.) P >0.05.

FIGS. 9A-C. HTTQ94 mediated ferroptosis upon ROS independent of GPX4. (A) Cell death assay. HTTQ94 Tet-on SK-N-BE(2)C control crispr and four independent ACSL4 crispr clones pre-incubated with doxycycline (0.5 μg/ml) for 16 h were treated with TBH (350 μM) for 24 h with/without Ferr-1 (2 μM). (B) Western blot analysis of ACSL4 and GPX4 in different ACSL4/GPX4 double crispr subclones from the HTTQ94 Tet-on SK-N-BE(2)C cell line. (C) Cell death assay. HTTQ94 Tet-on SK-N-BE(2)C control crispr and four independent ACSL4/GPX4 double crispr clones pre-incubated with doxycycline (0.5 μg/ml) for 16 h were treated with TBH (350 μM) for 24 h with/without Ferr-1 (2 μM). Cell death were calculated from three replicates; Data shown in (A) and (C) are means±SD. P-values were derived from two-tailed unpaired t-test. (n.s.) P >0.05.

FIGS. 10A-D. HTTQ94 mediated ferroptosis upon ROS independent of FSP1, DHODH and GCH1. (A) Q-PCR analysis of the knockdown efficiency of FSP1, GCH1 and DHODH in HTTQ94 Tet-on SK-N-BE(2)C cells transfected with control, FSP1, GCH1 or DHODH siRNA. (B) Western blot analysis of FSP1, GCH1 or DHODH in HTTQ94 Tet-on H1299 cells transfected with either Flag-FSP1, Flag-DHODH, Flag-GCH1 expressing plasmid or an empty vector. (C) Cell death assays for SK-N-BE(2)C knockdown cells. HTTQ94 Tet-on SK-N-BE(2)C cells transfected with control (ctrl), FSP1, GCH1 or DHODH siRNA were pre-incubated with doxycycline (0.5 μg/ml) for 16 h, then treated with TBH (350 μM) for additional 24 h. (D) Cell death assays for the overexpression cells. HTTQ94 Tet-on H1299 cells transfected with either Flag-FSP1, Flag-DHODH, Flag-GCH1 expressing plasmid or an empty vector were pre-incubated with doxycycline (0.5 μg/ml) for 16 h, then treated with TBH (50 μM) for additional 24 h. Cell death were calculated from three replicates; Data shown in (C) and (D) are are means±SD. P-values were derived from two-tailed unpaired t-test. (n.s.) P >0.05.

FIGS. 11A-F. Inactivation of ALOX5 abolished HTTQ94 mediated ferroptotic responses upon ROS-induced stress and glutamate. (A) Western blot analysis of ALOX5 in different ALOX5 crispr subclones from the HTTQ94 Tet-on SK-N-BE(2)C cell line. (B) Cell death assay. The HTTQ94 Tet-on SK-N-BE(2)C control crispr and four independent ALOX5 crispr cell lines pre-incubated with doxycycline (0.5 μg/ml) for 16 h were treated with TBH (350 μM) for 24 h with/without Ferr-1 (2 μM). (C) Cell death assay. The HTTQ94 Tet-on SK-N-BE(2)C control crispr and four independent ALOX5 crispr clones pre-incubated with doxycycline (0.5 μg/ml) for 16 h were treated with Erastin (40 μM) for 32 h with/without Ferr-1. (D) Quantification of lipid ROS levels from three replicates, related to main FIG. 4F. (E) Cell death assay. The HTTQ94 Tet-on HT-22 cells were treated with doxycycline (0.5 μg/ml) and glutamate (10 mM) in the presence or absence of Ferr-1(2 μM) or Zileuton (10 μM) for 20 h. (F) Q-PCR analysis of knockdown efficiency of ALOX5 in HTTQ94 Tet-on HT-22 cells, related to main FIG. 4G. Data shown in (B), (C), (D) and (E) are are means±SD. P-values were derived from two-tailed unpaired t-test. (***) P<0.001, (**) P≤0.01, (*) P≤0.05, (n.s.) P >0.05.

FIGS. 12A-D. The effect of ALOX5 and/or FLAP on HTTQ94-mediated ferroptosis. (A) Western blot analysis of V5-ALOX5 and HTTQ94 in HEK293 cells transfected with a V5-ALOX5 expressing plasmid and either an empty vector or indicated mHTT-GFP expressing vector. (B) Western Blot analysis for FLAP and HTTQ94 in HTTQ94 Tet-on SK-N-BE(2)C cells pre-incubated with/without doxycycline (0.5 μg/ml) for 16 h, and then treated with cycloheximide (CHX) at 200 μg/ml for the indicated times. (C) Q-PCR analysis of FLAP knockdown efficiency in HTTQ94 Tet-on SK-N-BE(2)C cells transfected with control siRNA or FLAP specific siRNA. (D) Cell death assay. HTTQ94 Tet-on SK-N-BE(2)C cells pre-incubated with doxycycline (0.5 μg/ml) for 16 h were treated with TBH (400 μM) for 24 h in the presence or absence of Ferr-1 (2 μM), Zileuton (10 μM) or MK886 (10 μM) for 24 h. Data shown in (D) are means±SD. P-values were derived from two-tailed unpaired t-test. (***) P≤0.001.

FIGS. 13A-D. TfR1 staining is able to specifically recognize ferroptotic cells induced by HTTQ94 expression. (A) Limb clasping analysis in HD and HD/Alox5null mice (9-, 11- and 13-week-old). p<0.01, n=7. (B-D), TfR1 staining in HT-22 HTTQ94 inducible cells upon TBH treatment. HT-22 HTTQ94 inducible cells were treated 1 μg/ml doxycycline O/N to induce HTTQ94 expression, followed by treatment with 30 μM TBH for 6 h, and then cells were stained with TfR1 antibody. (B) Representative images of TfR1 staining. (C) Quantification of TfR1 staining. (D) Cell death assay, related to panel B. Data shown in (A) are means±SD. Data shown in (C) and (D) are means±SEM. P-values were derived from two-tailed unpaired t-test. (***) P<0.001, (**) P≤0.01, (*) P<0.05, (n.s.) P >0.05.

### DETAILED DESCRIPTION

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

The singular forms “a”, “an” and “the” include plural reference unless the context clearly dictates otherwise.

As used herein the term “about” is used herein to mean approximately, roughly, around, or in the region of. When the term “about” is used in conjunction with a numerical range, it modifies that range by extending the boundaries above and below the numerical values set forth.

As used herein the term “variant” covers nucleotide or amino acid sequence variants which have about 99%, about 98%, about 97%, about 96%, about 95%, about 94%, about 93%, about 92%, about 91%, about 90%, about 85%, about 80%, about 75%, about 70%, or about 65% nucleotide identity, or about 99%, about 98%, about 97%, about 96%, about 95%, about 94%, about 93%, about 92%, about 91%, about 90%, about 85%, about 80%, about 75%, about 70%, or about 65% amino acid identity, including but not limited to variants comprising conservative, or non-conservative substitutions, deletions, insertions, duplications, or any other modification. The term variant as used herein includes functional and non-functional variants, and variants with reduced or altered activity.

The present disclosure provides compositions and methods for treating and/or preventing Huntington's disease (HD). Although it is well established that Huntington's disease (HD) is mainly caused by polyglutamine expanded mutant huntingtin (mHTT), the molecular mechanism of mHTT-mediated actions is not fully understood. Described herein is the discovery that expression of the N-terminal fragment containing the expanded polyglutamine (HTTQ94) of mHTT is able to promote both the ACSL4-dependent and the ACSL4-independent ferroptosis. Surprisingly, inactivation of the ACSL4-dependent ferroptosis fails to show any effect on the life span of Huntington's disease mouse.

Described herein is the discovery that ALOX5 mediates the ACSL4-independent ferroptosis induced by HTTQ94 via the stabilization of FLAP. By using RNAi-mediated screening, ALOX5 was identified as a major factor required for the ACSLA-independent ferroptosis induced by HTTQ94. Although ALOX5 is not required for the ferroptotic responses triggered by common ferroptosis inducers such as erastin, loss of ALOX5 expression abolishes HTTQ94-mediated ferroptosis upon reactive oxygen species (ROS)-induced stress. Interestingly, ALOX5 is also required for HTTQ94-mediated ferroptosis in neuronal cells upon high levels of glutamate. Mechanistically, HTTQ94 activates ALOX5-mediated ferroptosis by stabilizing FLAP, an essential cofactor of ALOX5-mediated lipoxygenase activity. Notably, inactivation of the Alox5 gene abrogates the ferroptosis activity in the striatal neurons from the HD mice; more importantly, loss of ALOX5 significantly ameliorates the pathological phenotypes and extends the life spans of these HD mice. Taken together, these results demonstrate that ALOX5 is critical for mHTT-mediated ferroptosis and suggest that ALOX5 is a promising new target for Huntington's disease. Moreover, mHTT-mediated ferroptosis is effectively blocked by Zileuton, a specific inhibitor of ALOX5. Silencing RNA (siRNA) can downregulate gene expression of specific genes (Agrawal N, Dasaradhi PVN, Mohmmed A, Malhotra P, Bhatnagar R K, Mukherjee S K. RNA Interference: Biology, Mechanism, and Applications. Microbial Mol Biol Rev. 2003 Dcc; 67(4): pp. 657-685).

As used herein mHTT refers to polyglutamine-expanded mutant huntingtin. Huntington's disease is caused by a CAG trinucleotide repeat expansion in exon 1 of the HTT gene, which results in an expanded polyglutamine (polyQ) tract in the encoded huntingtin protein, referred to as mutant huntingtin (mHTT).

As used herein HTTQ94 refers to the terminal fragment containing the expanded polyglutami. It is the mHTT fragment with the expanded 94 glutamine residues.

As used herein ALOX5 refers to arachidonate 5-lipoxygenase. Information on ALOX5 is available at www.ncbi.nlm.nih.gov/gene/240, the content of which is hereby incorporated by reference in its entirety include the nucleic acid and amino acid sequences of ALOX5.

As used herein FLAP refers to arachidonate 5-lipoxygenase activating protein. Information on FLAP is available at www.ncbi.nlm.nih.gov/gene/241, the content of which is hereby incorporated by reference in its entirety include the nucleic acid and amino acid sequences of FLAP.

### Methods of Treatment or Prevention

In certain aspects, described herein is the use of small interfering RNA (siRNA) platforms to silence the arachidonate 5-lipoxygenase (ALOX5) or 5-lipoxygenase-activating protein (FLAP). Also described herein is the use of ALOX5, FLAP, or both as a biomarker for diagnosing Huntington's disease.

The disclosure further provides methods for treating and/or preventing Huntington's disease in patients possessing increased expression of ALOX5, FLAP, or both. The compositions and methods disclosed herein can be used as disease modifying therapies to enable prevention or treatment of Huntington's disease and related disorders earlier in disease progression and improve clinical outcomes. The disclosure is based, at least in part, on the discovery, that ALOX5 expression is essential for HTTQ94-mediated ferroptosis, and that HTTQ94 promotes ALOX5-mediated ferroptosis by interacting with FLAP and up-regulating its stability. Described herein are methods of treatment or preventing Huntington's disease comprising either silencing or reducing expression of ALOX5, FLAP, or both among subjects who have high ALOX5 or FLAP expression compared to a subject who is not suffering from Huntington's disease.

In certain aspects, described herein is a method of treating or preventing Huntington's disease in a subject in need thereof, comprising administering to said subject a composition that targets the acyl-CoA synthetase long-chain family member 4 (ACSL4)-independent ferroptosis pathway.

In certain aspects, described herein is a method of treating or preventing Huntington's disease in a subject in need thereof, comprising: (i) identifying the subject as having an expression of mutant huntingtin (mHTT), and (ii) administering to said subject a composition that targets the acyl-CoA synthetase long-chain family member 4 (ACSL4)-independent ferroptosis pathway.

In certain aspects, described herein is a method of treating or preventing Huntington's disease in a subject in need thereof, comprising: (i) identifying the subject as having an increased expression level of arachidonate 5-lipoxygenase (ALOX5), and (ii) administering to said subject a composition that targets the acyl-CoA synthetase long-chain family member 4 (ACSL4)-independent ferroptosis pathway.

In certain aspects, described herein is a method of treating or preventing Huntington's disease in a subject in need thereof, comprising: (i) identifying the subject as having an increased expression level of 5-lipoxygenase-activating protein (FLAP), and (ii) administering to said subject a composition that targets the acyl-CoA synthetase long-chain family member 4 (ACSL4)-independent ferroptosis pathway.

In some embodiments, the subject expresses mHTT, an increased level of ALOX5, or an increased level of FLAP or a combination thereof. In some embodiments, expression of mHTT is determined from a sample from the subject. In some embodiments, expression level of ALOX5 and/or FLAP is determined from a sample from the subject. In some embodiments, expression or expression level is a protein level, a mRNA expression level, or combination thereof. Methods for detecting and quantifying ALOX5 or FLAP mRNA in biological samples are known the art. In some embodiments, the level of mRNA is determined using RT-qPCR or RNA-seq.

In one embodiment, a sample comprises, a blood sample, serum, cells (including whole cells, cell fractions, cell extracts, and cultured cells or cell lines), tissues (including tissues obtained by biopsy), body fluids (e.g., urine, sputum, amniotic fluid, synovial fluid), or from media (from cultured cells or cell lines). In some embodiments, the sample is blood or blood serum or CSF. In some embodiments, the same is cells of the subject. In some embodiments, the sample is nervous system tissue of the subject. In some embodiments, the sample comprises brain cells of the subject.

In some embodiments, the subject expressing mHTT further expresses increased level of ALOX5, or an increased level of FLAP or a combination thereof. In some embodiments, the subject expressing an increased level of ALOX5 further expresses mHTT, or an increased level of FLAP or a combination thereof. In some embodiments, the subject expressing an increased level of FLAP further expresses mHTT, or an increased level of ALOX5 or a combination thereof. In some embodiments, the increased levels are ALOX5 mRNA levels, ALOX5 protein levels, or both ALOX5 mRNA levels and ALOX5 protein levels in a sample from the subject. In some embodiments, the increased levels are FLAP mRNA levels, FLAP protein levels, or both FLAP mRNA levels and FLAP protein levels in a sample from the subject. In some embodiments, ALOX5 or FLAP mRNA levels and ALOX5 or FLAP protein levels the from a sample from the subject.

In some embodiments, the composition inhibits or reduces ALOX5 expression in the subject. In some embodiments, the composition inhibits or reduces FLAP expression in the subject.

In some embodiments, the composition reduces ALOX5 expression in the subject compared to ALOX5 expression in a subject suffering from Huntington's disease or compared to ALOX5 expression in the subject before administration of the composition. ALOX5 expression may be reduced using any known method in the art. The expression of ALOX5 may be reduced by at least about 5% to about 95%, e.g., about 5%, 10%, 15%, 20%, 30%, 40%, 50%, 60%, 70%, 80%, 90%, 95%, relative to corresponding expression of ALOX5 in a subject suffering from Huntington's disease or in the subject before administration of the composition. The expression of ALOX5 may be silenced relative to corresponding expression of ALOX5 in a subject suffering from Huntington's disease or in the subject before administration of the composition. In some embodiments, the expression of ALOX5 is reduced cells of the subject.

In some embodiments, the composition reduces FLAP expression in the subject compared to FLAP expression in a subject suffering from Huntington's disease or compared to FLAP expression before administration of the composition. FLAP expression may be reduced using any known method in the art. In some embodiments, the composition inhibits or reduces ALOX5 expression in the subject. In some embodiments, the composition inhibits or reduces FLAP expression in the subject. The expression of FLAP may be reduced by at least about 5% to about 95%, e.g., about 5%, 10%, 15%, 20%, 30%, 40%, 50%, 60%, 70%, 80%, 90%, 95%, relative to corresponding expression of FLAP in a subject suffering from Huntington's disease or in the subject before administration of the composition. The expression of FLAP may be silenced relative to corresponding expression of FLAP in a subject suffering from Huntington's disease or in the subject before administration of the composition. In some embodiments, the expression of FLAP is reduced cells of the subject.

In some embodiments, the composition comprises Zileuton. Zileuton is an FDA-approved orally active inhibitor of ALOX5 used for the maintenance treatment of asthma. Zileuton has the chemical name (+)-1 (1-Benzo[b]thien-2-ylethyl)-1-hydroxyurea and the chemical structure below. In some embodiments, the composition comprises MK.886. MK.886 is a cell-permeable and orally active FLAP Inhibitor. MK.886 has the chemical structure below.

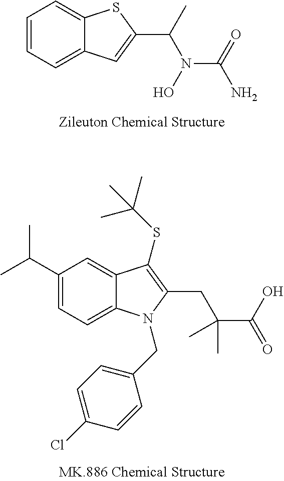 [SMILES: CC(c1cc2ccccc2s1)N(O)C(N)=O ; CC(C)c1ccc2c(c1)c(SC(C)(C)C)c(CC(C)(C)C(=O)O)n2Cc1ccc(Cl)cc1]

In some embodiments, the composition comprises docebenone (AA 861). is a potent, selective and orally active 5-LO (5-lipoxygenase) inhibitor. In some embodiments, the composition comprises

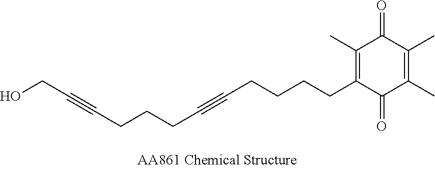 [SMILES: CC1=C(C)C(=O)C(CCCCC#CCCCC#CCO)=C(C)C1=O]

In some embodiments, the composition comprises boswellic acids. 11-keto-boswellic acids inhibit ALOX5. In some embodiments, the composition comprises atreleuton (ABT-761 or VIA-2291). Atreleuton is a selective, reversible, and orally bioavailable ALOX5 inhibitor. In some embodiments, the composition comprises

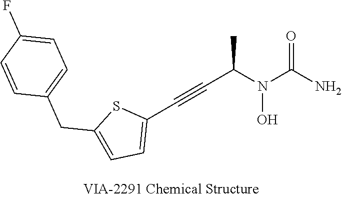 [SMILES: C[C@H](C#Cc1ccc(Cc2ccc(F)cc2)s1)N(O)C(N)=O]

In some embodiments, the composition comprises setileuton (1,3,4-oxadiazole MK-0633). Setileuton (1,3,4-oxadiazole MK-0633), a potent and selective ALOX5 inhibitor. In some embodiments, the composition comprises

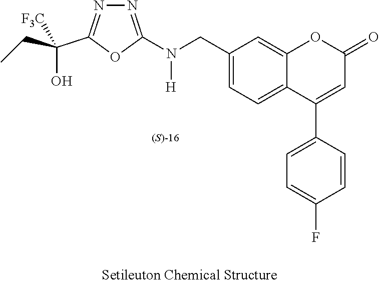 [SMILES: CC[C@](O)(c1nnc(NCc2ccc3c(-c4ccc(F)cc4)cc(=O)oc3c2)o1)C(F)(F)F]

In some embodiments, the composition comprises PF-4191834 or CJ-13610. In some embodiments, the composition comprises

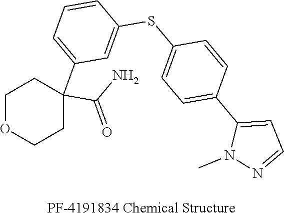 [SMILES: Cn1nccc1-c1ccc(Sc2cccc(C3(C(N)=O)CCOCC3)c2)cc1]

In some embodiments, the composition comprises flavocoxid. Flavocoxid is a mixture of purified bioflavonoids that inhibits COX-1, COX-2, and ALOX5 in vitro and in animal models.

In some embodiments, the composition comprises a ALOX5 small interfering ribonucleic acid (siALOX5). For example, in various embodiments the composition is a vector comprising at least one of the small interfering ribonucleic acid (siRNA) designs targeting ALOX5. In various embodiments, the siALOX5 is at least 75%, 80%, 85%, 90%, 95%, 96%, 97%, 98% or 99% identical to the siRNA designs of ALOX5. In some embodiments, the composition comprises a FLAP small interfering ribonucleic acid (siFLAP). For example, in various embodiments the composition is a vector comprising at least one of the small interfering ribonucleic acid (siRNA) designs targeting FLAP. In various embodiments, the siRNA is at least 75%, 80%, 85%, 90%, 95%, 96%, 97%, 98% or 99% identical to the siRNA designs of FLAP. In some embodiments, the siRNA comprises one or more gene-specific siRNAs (e.g. siRNA targeting ALOX5 or FLAP) as a pooled siRNA. In some embodiments, the siRNA comprises two or more gene-specific siRNAs (e.g. siRNA targeting ALOX5 or FLAP) as a pooled siRNA. In some embodiments, the siRNA comprises three or more gene-specific siRNAs (e.g. siRNA targeting ALOX5 or FLAP) as a pooled siRNA. In some embodiments, the siRNA comprises four or more gene-specific siRNAs (e.g. siRNA targeting ALOX5 or FLAP) as a pooled siRNA. In some embodiments, the siRNA comprises four gene-specific siRNAs (e.g. siRNA targeting ALOX5 or FLAP) as a pooled siRNA. Exemplary pooled siRNA is a SMARTpool siRNA (Dharmacon, Horizon Discovery). Different siRNAs formats, which in some embodiments are included as pooled siRNAs, are known in the art, including but not limited to siRNA with chemical modifications. Exemplary siRNA formats include ON-TARGETplus siRNA, Accell siRNA, siGENOME siRNA, or Lincode siRNA (Dharmacon, Horizon Discovery).

In some embodiments, the composition comprises a ALOX5 short-hairpin ribonucleic acid (shALOX5). For example, in various embodiments the composition is a nucleic acid vector encoding at least one of the shRNA designs targeting ALOX5. In various embodiments, the shRNA comprises a nucleic acid sequence at least 75%, 80%, 85%, 90%, 95%, 96%, 97%, 98% or 99% identical to the nucleotide sequence of the siRNA designs of ALOX5. In some embodiments, the composition comprises a FLAP short-hairpin ribonucleic acid (shFLAP). For example, in various embodiments the composition is a nucleic acid vector encoding at least one of the shRNA designs targeting FLAP. In various embodiments, the shRNA comprises a nucleic acid sequence at least 75%, 80%, 85%, 90%, 95%, 96%, 97%, 98% or 99% identical to the nucleotide sequence of the siRNA designs of FLAP.

In some embodiments, the composition comprises a guide RNA or a single-molecule guide RNA comprising a spacer sequence that is complementary to a portion of a nucleic acid sequence encoding ALOX5. In some embodiments, the composition comprises a guide RNA or a single-molecule guide RNA comprising a spacer sequence that is complementary to a portion of a nucleic acid sequence encoding FLAP. In some embodiments, the guide RNA or the single molecule guide RNA is pre-complexed with a DNA endonuclease. In some embodiments, the DNA endonuclease is a Cas9 or dCas9 endonuclease.

In some embodiments, the composition comprises a vector. In some embodiments, the vector is a viral vector wherein the viral vector encapsulates a nucleic acid encoding the shALOX5, a nucleic acid encoding the shFLAP, a nucleic acid encoding the guide ribonucleic acid or a single molecule guide RNA comprising a spacer sequence that is complementary to a portion of a nucleic acid sequence encoding ALOX5, or a nucleic acid encoding the guide ribonucleic acid or a single molecule guide RNA comprising a spacer sequence that is complementary to a portion of a nucleic acid sequence encoding FLAP. In some embodiments, the viral vector is an adeno-associated vector (AAV). In various embodiments the AAV is AAV 1, 2, 3, 4, 5, 6, 7, 8, 9, or 10 or variants thereof.

In some embodiments, the subject is a mammal. In some embodiments, the mammal is a human. In some embodiments, the human subject has an increased expression level of ALOX5 compared to a subject not suffering from Huntington's disease. For example, expression of ALOX5 of a subject may be increased by at least 1.5 times greater than expression of ALOX5 of a subject not suffering from Huntington's disease. Expression of ALOX5 of a subject may be increased by at least 3 times greater than expression of ALOX5 of a subject not suffering from Huntington's disease. Expression of ALOX5 of a subject may be increased by at least 5 times greater than expression of ALOX5 of a subject not suffering from Huntington's disease. Expression of ALOX5 of a subject may be increased by at least 10 times greater than expression of ALOX5 of a subject not suffering from Huntington's disease.

In some embodiments, the human subject has an increased expression level of FLAP compared to a subject not suffering from Huntington's disease. For example, expression of FLAP of a subject may be increased by at least 1.5 times greater than expression of FLAP of a subject not suffering from Huntington's disease. Expression of FLAP of a subject may be increased by at least 3 times greater than expression of FLAP of a subject not suffering from Huntington's disease. Expression of FLAP of a subject may be increased by at least 5 times greater than expression of FLAP of a subject not suffering from Huntington's disease. Expression of FLAP of a subject may be increased by at least 10 times greater than expression of FLAP of a subject not suffering from Huntington's disease.

In some embodiments, a population of cells can be contacted with a compound or agent which, for example, includes subjecting the cells to an appropriate culture media which comprises the indicated compound or agent. Where the cell population is in vivo, contacting the cell population includes administering the compound or agent in a pharmaceutical composition to a subject via an appropriate administration route such that the compound or agent contacts the cell population in vivo.

For in vivo methods, a therapeutically effective amount of a compound described herein can be administered to a subject. Methods of administering compounds to a subject are known in the art and easily available to one of skill in the art.

As described herein, the methods of treatment described herein refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. Methods described herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom.

A therapeutically effective amount of an agent or composition disclosed herein, for example, is one that is effective for preventing, ameliorating, treating or delaying the onset of a disease or condition.

The pharmaceutical compositions of the inventions can be administered to any animal that can experience the beneficial effects of the agents of the invention. Such animals include humans and non-humans such as primates, pets and farm animals.

The present invention also comprises pharmaceutical compositions comprising the agents disclosed herein. Routes of administration and dosages of effective amounts of the pharmaceutical compositions comprising the agents are also disclosed. The agents of the present invention can be administered in combination with other pharmaceutical agents in a variety of protocols for effective treatment of disease.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the viral vectors, siRNA, RNAi, shRNA or other inhibitors, or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the brain, and more specifically neuronal cells. The liposomes will be targeted to and taken up selectively by the desired tissue. Also included in a targeted drug delivery system is nanoparticle specific brain delivery of the viral vectors, siRNA, RNAi, shRNA or other inhibitors, or compound, alone or in combination with similar compounds. A summary of various delivery methods and techniques of siRNA administration in ongoing clinical trials is provided in Zuckerman and Davis 2015; Nature Rev. Drug Discovery, Vol. 14: 843-856, December 2015 the contents of which is hereby incorporated by reference in its entirety.

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosages can also be administered in a patient-specific manner to provide a predetermined concentration of the agents in the blood, as determined by techniques accepted and routine in the art.

ALOX5 or FLAP expression may be reduced using any known method in the art. For example, in various embodiments the composition is a viral vector encoding at least one of the siRNA designs targeting ALOX5 or FLAP. In various embodiments, the vector is a viral vector. In various embodiments, the viral vector is an AAV vector. In various embodiments, the viral vector is a vector that preferentially targets the brain or brain cells. In various embodiments the AAV is AAV 1, 2, 3, 4, 5, 6, 7, 8, 9, or 10 or variants thereof. In various embodiments, the subject is a mammal. In various embodiments, the mammal is a human. In various embodiments, the human has increased expression of ALOX5 or FLAP compared to a human not suffering from Huntington's disease.

In certain aspects, described herein is small interfering ribonucleic acid (siRNA) targeting the acyl-CoA synthetase long-chain family member 4 (ACSLA)-independent ferroptosis pathway. In some embodiments, the siRNA is an ALOX5 small interfering ribonucleic acid (siALOX5). In some embodiments, the siRNA is a FLAP small interfering ribonucleic acid (siFLAP). In certain aspects, described herein is a vector comprising a siRNA.

In certain aspects, described herein is a nucleic acid comprising a sequence encoding a short hairpin ribonucleic acid (shRNA) targeting the acyl-CoA synthetase long-chain family member 4 (ACSL4)-independent ferroptosis pathway. In certain aspects, described herein is a vector comprising any of the nucleic acid described above. In certain aspects, described herein is a viral vector comprising the nucleic acid described above. In some embodiments, the viral vector is an AAV vector.

In some embodiment, targeted gene expression can be reduced by several genome editing techniques such as RNAi (RNA interference), zinc finger nucleases (ZFNs), a TALE-effector domain nuclease (TALLEN), CRISPR/Cas9 systems which are known in the art. In some embodiment, the CRISPR/Cas9 systems comprise a guide RNA (gRNA) or a single-molecule guide RNA (sgRNA). In some embodiment, the gRNA or sgRNA comprises a spacer sequence that is complementary to a portion of a nucleic acid sequence encoding ALOX family. In some embodiment, the gRNA or sgRNA comprises a spacer sequence that is complementary to a portion of a nucleic acid sequence encoding FLAP.

Inhibition of RNA encoding ALOX5 or FLAP can effectively modulate the expression of these proteins. Inhibitors can include siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding an EGFR fusion molecule can be synthesized, e.g., by conventional phosphodiester techniques. Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector. Methods for producing and testing dsRNA or siRNA molecules are known in the art.

RNA polymerase III transcribed DNAs contain promoters, such as the U6 promoter. These DNAs can be transcribed to produce small hairpin RNAs in the cell that can function as siRNA or linear RNAs, which can function as antisense RNA. The ALOX5 or FLAP inhibitor can comprise ribonucleotides, deoxyribonucleotides, synthetic nucleotides, or any suitable combination such that the target RNA and/or gene is inhibited. In addition, these forms of nucleic acid can be single, double, triple, or quadruple stranded.

In some embodiments, the compositions is delivered through the blood brain barrier (BBB). Different methods for delivering compositions through the BBB, are known in the art, including but not limited to altering BBB integrity or osmotic disruption, conjugation to a biological carrier, use of shuttle compounds, use of stimuli responsive nanoparticles, hyperosmolar solutions, microbubbles and focused ultrasound (FUS), or receptor-mediated transcytosis (RMT).

### Pharmaceutical Composition

In certain aspects, described herein is a composition for treating or preventing Huntington's disease, comprising an expression vector capable of targeting the ACSL4-independent ferroptosis pathway. In some embodiments, the composition reduces arachidonate 5-lipoxygenase (ALOX5) expression in a subject in need thereof. The expression of ALOX5 may be reduced by at least about 5% to about 95%, e.g., about 5%, 10%, 15%, 20%, 30%, 40%, 50%, 60%, 70%, 80%, 90%, 95%, relative to corresponding expression of ALOX5 in a subject suffering from Huntington's disease or in the subject before administration of the composition. The expression of ALOX5 may be silenced relative to corresponding expression of ALOX5 in a subject suffering from Huntington's disease or in the subject before administration of the composition. In some embodiments, the expression of ALOX5 is reduced cells of the subject.

In some embodiments, the composition reduces 5-lipoxygenase-activating protein (FLAP) expression in a subject in need thereof. The expression of ALOX5 may be reduced by at least about 5% to about 95%, e.g., about 5%, 10%, 15%, 20%, 30%, 40%, 50%, 60%, 70%, 80%, 90%, 95%, relative to corresponding expression of FLAP in a subject suffering from Huntington's disease or in the subject before administration of the composition. The expression of FLAP may be silenced relative to corresponding expression of FLAP in a subject suffering from Huntington's disease or in the subject before administration of the composition. In some embodiments, the expression of FLAP is reduced cells of the subject.

In some embodiments, the composition comprises an ALOX5 small interfering ribonucleic acid (siALOX5). In some embodiments, the composition comprises a FLAP small interfering ribonucleic acid (siFLAP). In some embodiments, the composition comprises an ALOX5 short-hairpin ribonucleic acid (shALOX5). In some embodiments, the composition comprises a FLAP short-hairpin ribonucleic acid (shFLAP).

In some embodiments, the composition comprises a guide ribonucleic acid or a single molecule guide RNA comprising a spacer sequence that is complementary to a portion of a nucleic acid sequence encoding ALOX5. In some embodiments, the composition comprises a guide ribonucleic acid or a single molecule guide RNA comprising a spacer sequence that is complementary to a portion of a nucleic acid sequence encoding FLAP. In some embodiments, the guide RNA or the single molecule guide RNA is pre-complexed with a DNA endonuclease. In some embodiments, the DNA endonuclease is a Cas9 or dCas9 endonuclease.

In certain aspects, the composition further comprises a vector comprising the siRNA or shRNA or gRNA or sgRNAs described herein. In some embodiments, the composition further comprises a viral vector wherein the viral vector encapsulates a nucleic acid encoding the shALOX5, a nucleic acid encoding the shFLAP, a nucleic acid encoding the guide ribonucleic acid or a single molecule guide RNA comprising a spacer sequence that is complementary to a portion of a nucleic acid sequence encoding ALOX5, or a nucleic acid encoding the guide ribonucleic acid or a single molecule guide RNA comprising a spacer sequence that is complementary to a portion of a nucleic acid sequence encoding FLAP. In some embodiments, the viral vector is an AAV vector. In various embodiments the AAV is AAV 1, 2, 3, 4, 5, 6, 7, 8, 9, or 10 or variants thereof.

In certain aspects, described herein is small interfering ribonucleic acid (siRNA) targeting the acyl-CoA synthetase long-chain family member 4 (ACSLA)-independent ferroptosis pathway. In some embodiments, the siRNA is an ALOX5 small interfering ribonucleic acid (siALOX5). In some embodiments, the siRNA is a FLAP small interfering ribonucleic acid (siFLAP). In certain aspects, described herein is a vector comprising a SiRNA.

In certain aspects, described herein is a nucleic acid comprising a sequence encoding a short hairpin ribonucleic acid (shRNA) targeting the acyl-CoA synthetase long-chain family member 4 (ACSL4)-independent ferroptosis pathway. In certain aspects, described herein is a vector comprising any of the nucleic acid described above. In certain aspects, described herein is a viral vector comprising the nucleic acid described above. In some embodiments, the viral vector is an AAV vector. In various embodiments the AAV is AAV 1, 2, 3, 4, 5, 6, 7, 8, 9, or 10 or variants thereof.

In some embodiments, the composition comprises Zileuton. Zileuton is FDA approved for treatment of asthma. The FDA prescribing label is available at FDA.gov and is hereby incorporated by reference in its entirety.

In some embodiments, the composition comprises (±)-1 (1-Benzo[b]thien-2-ylethyl)-1-hydroxyurea or pharmaceutically acceptable salts thereof. In some embodiments, the composition comprises (±)-1 (1-Benzo[b]thien-2-ylethyl)-1-hydroxyurea. In some embodiments, the composition comprises

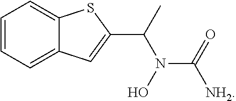 [SMILES: CC(c1cc2ccccc2s1)N(O)C(N)=O]

In some embodiments, the composition consists of (±)-1 (1-Benzo[b]thien-2-ylethyl)-1-hydroxyurea or pharmaceutically acceptable salts thereof. In some embodiments, the composition consists of (±)-1 (1-Benzo[b]thien-2-ylethyl)-1-hydroxyurea. In some embodiments, the composition consists of

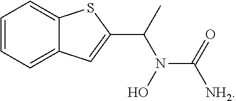 [SMILES: CC(c1cc2ccccc2s1)N(O)C(N)=O]

In some embodiments, the composition consists essentially of (±)-1 (1-Benzo[b]thien-2-ylethyl)-1-hydroxyurea or pharmaceutically acceptable salts thereof. In some embodiments, the composition consists essentially of (±)-1 (1-Benzo[b]thien-2-ylethyl)-1-hydroxyurea. In some embodiments, the composition consists essentially of

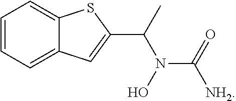 [SMILES: CC(c1cc2ccccc2s1)N(O)C(N)=O]

In some embodiments, the composition comprises a tablet comprising Zileuton. In some embodiments, the tablet is as an extended release tablet. In some embodiments, the composition comprises a tablet comprising 600 mg of Zileuton. In some embodiments, the composition comprises a tablet comprising 100 mg, 200 mg, 300 mg, 400 mg, 500 mg, 600 mg, 700 mg, 800 mg, 900 mg, 1000 mg, 1100 mg, or 1200 mg or more of Zileuton. In some embodiments, the composition is administered at a dose of 2400 mg daily. In some embodiments, the composition is administered at a dose of 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, or 3000 mg or more daily. In some embodiments, the composition is administered as two 600 mg tablets twice daily (or equivalents thereof e.g four 300 mg tablets twice daily). In some embodiments, the composition is administered within one hour after morning and evening meals.

In some embodiments, the composition comprises

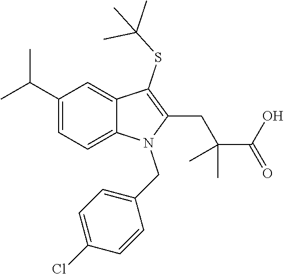 [SMILES: CC(C)c1ccc2c(c1)c(SC(C)(C)C)c(CC(C)(C)C(=O)O)n2Cc1ccc(Cl)cc1]

or pharmaceutically acceptable salts thereof. In some embodiments, the composition comprises

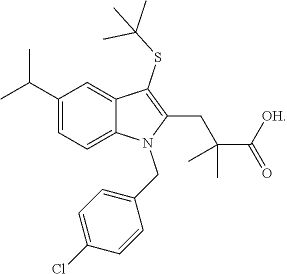 [SMILES: CC(C)c1ccc2c(c1)c(SC(C)(C)C)c(CC(C)(C)C(=O)O)n2Cc1ccc(Cl)cc1]

In some embodiments, the composition consists of

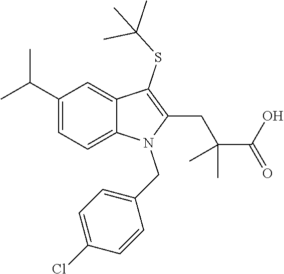 [SMILES: CC(C)c1ccc2c(c1)c(SC(C)(C)C)c(CC(C)(C)C(=O)O)n2Cc1ccc(Cl)cc1]

or pharmaceutically acceptable salts thereof. In some embodiments, the composition consists of

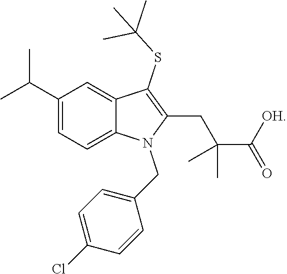 [SMILES: CC(C)c1ccc2c(c1)c(SC(C)(C)C)c(CC(C)(C)C(=O)O)n2Cc1ccc(Cl)cc1]

In some embodiments, the composition consists essentially of

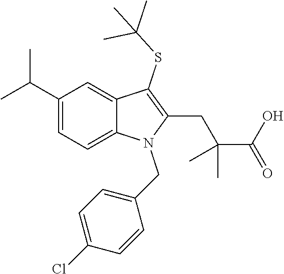 [SMILES: CC(C)c1ccc2c(c1)c(SC(C)(C)C)c(CC(C)(C)C(=O)O)n2Cc1ccc(Cl)cc1]

or pharmaceutically acceptable salts thereof. In some embodiments, the composition consists essentially of

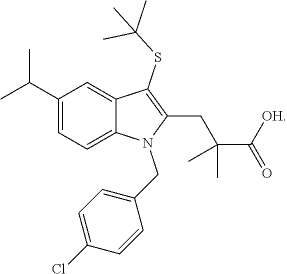 [SMILES: CC(C)c1ccc2c(c1)c(SC(C)(C)C)c(CC(C)(C)C(=O)O)n2Cc1ccc(Cl)cc1]

In some embodiments, the composition comprises docebenone (AA 861). is a potent, selective and orally active 5-LO (5-lipoxygenase) inhibitor. In some embodiments, the composition comprises

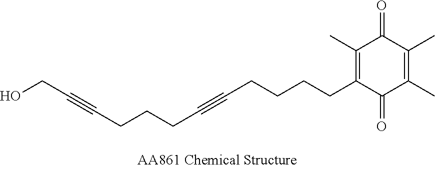 [SMILES: CC1=C(C)C(=O)C(CCCCC#CCCCC#CCO)=C(C)C1=O]

In some embodiments, the composition comprises boswellic acids. 11-keto-boswellic acids inhibit ALOX5. In some embodiments, the composition comprises atreleuton (ABT-761 or VIA-2291). Atreleuton is a selective, reversible, and orally bioavailable ALOX5 inhibitor. In some embodiments, the composition comprises

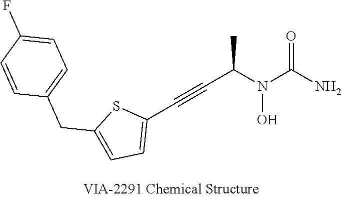 [SMILES: C[C@H](C#Cc1ccc(Cc2ccc(F)cc2)s1)N(O)C(N)=O]

In some embodiments, the composition comprises setileuton (1,3,4-oxadiazole MK-0633). Setileuton (1,3,4-oxadiazole MK-0633), a potent and selective ALOX5 inhibitor. In some embodiments, the composition comprises

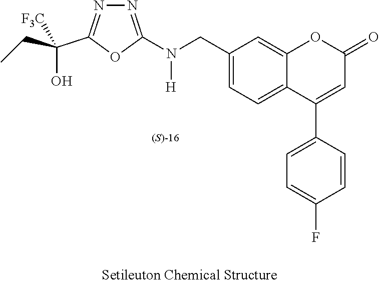 [SMILES: CC[C@](O)(c1nnc(NCc2ccc3c(-c4ccc(F)cc4)cc(=O)oc3c2)o1)C(F)(F)F]

In some embodiments, the composition comprises PF-4191834 or CJ-13610. In some embodiments, the composition comprises

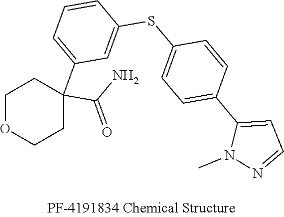 [SMILES: Cn1nccc1-c1ccc(Sc2cccc(C3(C(N)=O)CCOCC3)c2)cc1]

In some embodiments, the composition comprises flavocoxid. Flavocoxid is a mixture of purified bioflavonoids that inhibits COX-1, COX-2, and ALOX5 in vitro and in animal models. Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the viral vectors, siRNA, RNAi, shRNA or other inhibitors, or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the brain, and more specifically neuronal cells. The liposomes will be targeted to and taken up selectively by the desired tissue. Also included in a targeted drug delivery system is nanoparticle specific brain delivery of the viral vectors, siRNA, RNAi, shRNA or other inhibitors, or compound, alone or in combination with similar compounds. A summary of various delivery methods and techniques of siRNA administration in ongoing clinical trials is provided in Zuckerman and Davis 2015; Nature Rev. Drug Discovery, Vol. 14: 843-856, December 2015 the contents of which is hereby incorporated by reference in its entirety.

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosages can also be administered in a patient-specific manner to provide a predetermined concentration of the agents in the blood, as determined by techniques accepted and routine in the art.

In various embodiments, the present application discloses compositions for regulating the ferroptosis through the acyl-CoA synthetase long-chain family member 4 (ACSL4)-independent ferroptosis pathway. In various embodiments, the present application discloses a composition that reduces ALOX5 expression or function. In various embodiments, the present application discloses a composition that inhibits FLAP expression or function. In various embodiments, the present application discloses a composition comprising ALOX5 siRNA. In various embodiments, the present application discloses a composition comprising FLAP siRNA. For example, in various embodiments the composition is a vector comprising at least one of the small interfering ribonucleic acid (siRNA) designs targeting ALOX5 or FLAP. In various embodiments, the siRNA is at least 75%, 80%, 85%, 90%, 95%, 96%, 97%, 98% or 99% identical to the siRNA designs of ALOX5 or FLAP. In various embodiments, the vector is a viral vector. In various embodiments, the viral vector is an AAV vector. In various embodiments, the viral vector is a vector that preferentially targets the brain or brain cells. In various embodiments the AAV is AAV 1, 2, 3, 4, 5, 6, 7, 8, 9, or 10 or variants thereof.

In various embodiments, the present application discloses compositions for regulating the ferroptosis through the acyl-CoA synthetase long-chain family member 4 (ACSL4)-independent ferroptosis pathway. In various embodiments, the present application discloses a composition that reduces ALOX5 expression or function. In various embodiments, the present application discloses a composition that inhibits FLAP expression or function. In various embodiments, the present application discloses a composition comprising ALOX5 siRNA. In various embodiments, the present application discloses a composition comprising FLAP siRNA. For example, in various embodiments the composition is a vector comprising at least one of the small interfering ribonucleic acid (siRNA) designs targeting ALOX5 or FLAP. In various embodiments, the siRNA is at least 75%, 80%, 85%, 90%, 95%, 96%, 97%, 98% or 99% identical to the siRNA designs targeting ALOX5 or FLAP. In various embodiments, the vector is a viral vector. In various embodiments, the viral vector is an AAV vector. In various embodiments, the viral vector is a vector that preferentially targets the brain or brain cells. In various embodiments the AAV is AAV 1, 2, 3, 4, 5, 6, 7, 8, 9, or 10 or variants thereof.

### Method of Diagnosis

In certain aspects, the invention provides a method of diagnosing Huntington's disease in a subject comprising, determining the presence of mutant huntingtin (mHTT) in the subject. In certain aspects, the invention provides a method of diagnosing Huntington's disease in a subject comprising (i) determining the level of arachidonate 5-lipoxygenase (ALOX5) in the subject; and (ii) diagnosing the subject with Huntington's disease if the level of ALOX5 expression in the subject is increased as compared to a subject not suffering from Huntington's disease. For example, expression of ALOX5 of a subject may be increased by at least 1.5 times greater than expression of ALOX5 of a subject not suffering from Huntington's disease. Expression of ALOX5 of a subject may be increased by at least 3 times greater than expression of ALOX5 of a subject not suffering from Huntington's disease. Expression of ALOX5 of a subject may be increased by at least 5 times greater than expression of ALOX5 of a subject not suffering from Huntington's disease. Expression of ALOX5 of a subject may be increased by at least 10 times greater than expression of ALOX5 of a subject not suffering from Huntington's disease. In some embodiments, the method further comprises determining the level of FLAP in the subject. In some embodiments, the method further comprises determining the whether the subject expresses mHTT. In certain aspects, the invention provides a method of diagnosing Huntington's disease in a subject comprising (i) determining determining the level of 5-lipoxygenase-activating protein (FLAP) in the subject; and (ii) diagnosing the subject with Huntington's disease if the level of FLAP expression in the subject is increased as compared to a subject not suffering from Huntington's disease. For example, expression of FLAP of a subject may be increased by at least 1.5 times greater than expression of FLAP of a subject not suffering from Huntington's disease. Expression of FLAP of a subject may be increased by at least 3 times greater than expression of FLAP of a subject not suffering from Huntington's disease. Expression of FLAP of a subject may be increased by at least 5 times greater than expression of FLAP of a subject not suffering from Huntington's disease. Expression of FLAP of a subject may be increased by at least 10 times greater than expression of FLAP of a subject not suffering from Huntington's disease. In some embodiments, the method further comprises determining the level of ALOX5 in the subject. In some embodiments, the method further comprises determining whether the subject expresses mHTT.

In some embodiments the level of ALOX5, FLAP, and/or mHTT is determined from a sample from the subject. In some embodiments, Huntington disease is diagnosed in the subject by determining ALOX5 mRNA levels, ALOX5 protein levels, or both ALOX5 mRNA levels and ALOX5 protein levels in a sample from the subject. In some embodiments, Huntington disease is diagnosed in the subject by determining FLAP mRNA levels, FLAP protein levels, or both FLAP mRNA levels and FLAP protein levels in a sample from the subject. In some embodiments, the subject is diagnosed with Huntington's disease if the level of both FLAP and ALOX5 expression in the subject is increased as compared to a subject not suffering from Huntington's disease. In some embodiments, the subject is diagnosed with Huntington's disease if the level of FLAP expression in the subject is increased as compared to a subject not suffering from Huntington's disease and the subject expresses mHTT. In some embodiments, the subject is diagnosed with Huntington's disease if the level of ALOX5 expression in the subject is increased as compared to a subject not suffering from Huntington's disease and the subject expresses mHTT. In some embodiments, the subject is diagnosed with Huntington's disease if the level of both ALOX5 and FLAP expression in the subject is increased as compared to a subject not suffering from Huntington's disease and the subject expresses mHTT. In some embodiments, the invention comprises detecting in a biological sample whether there is an increase in an mRNA encoding ALOX5 or FLAP. Methods for detecting and quantifying ALOX5 or FLAP molecules in biological samples are known the art. For example, protocols for detecting and measuring a ALOX5 or FLAP protein molecule using either polyclonal or monoclonal antibodies specific for the polypeptide are well established. Non-limiting examples include Western blot, enzyme-linked immunosorbent assay (ELISA), radioimmunoassay (RIA), and fluorescence activated cell sorting (FACS). Methods for detecting and quantifying ALOX5 or FLAP mRNA in biological samples are known the art. In some embodiments, the level of mRNA is determined using RT-qPCR or RNA-seq.

In one aspect, the invention provides a device for determining whether a sample from a subject contains ALOX5 or FLAP protein, or a combination thereof, the device comprising at least one antibody that specifically binds to ALOX5 or FLAP protein, or a fragment thereof. In another aspect, the invention provides a device for determining whether a sample from a subject contains ALOX5 or FLAP nucleic acid, or a combination thereof, the device comprising at least one primer, primer pair, or nucleic acid probe, that specifically binds to ALOX5 or FLAP nucleic acid, or a fragment thereof.

In one embodiment, a biological sample comprises, a blood sample, serum, cells (including whole cells, cell fractions, cell extracts, and cultured cells or cell lines), tissues (including tissues obtained by biopsy), body fluids (e.g., urine, sputum, amniotic fluid, synovial fluid), or from media (from cultured cells or cell lines). In some embodiments, the sample is blood or blood serum or CSF. In some embodiments, the same is cells of the subject. In some embodiments, the sample is nervous system tissue of the subject. In some embodiments, the sample comprises brain cells of the subject. The methods of detecting or quantifying a ALOX5 or FLAP molecule include, but are not limited to, amplification-based assays with (signal amplification) hybridization based assays and combination amplification-hybridization assays. For detecting and quantifying a ALOX5 or FLAP molecule, an exemplary method is an immunoassay that utilizes an antibody or other binding agents that specifically bind to an ALOX5 or FLAP protein or epitope of such, for example, Western blot or ELISA assays. Methods for detecting and quantifying ALOX5 or FLAP mRNA in biological samples are known the art. In some embodiments, the level of mRNA is determined using RT-qPCR or RNA-seq.

In some embodiments, the compositions is delivered through the blood brain barrier (BBB). Different methods delivering compositions through the BBB, are known in the art, including but not limited to altering BBB integrity or osmotic disruption, conjugation to a biological carrier, use of shuttle compounds, use of stimuli responsive nanoparticles, hyperosmolar solutions, microbubbles and focused ultrasound (FUS), or receptor-mediated transcytosis (RMT).

### EXAMPLES

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield similar results.

### Example 1: HTTQ94 Expression Sensitizes Neuronal Cells and Other Cell Types to Ferroptosis

Although it is well-established that Huntington's disease (HD) is mainly caused by polyglutamine expanded mutant huntingtin (mHTT), the molecular mechanism of mHTT-mediated actions is not fully understood. Here, expression of the N-terminal fragment containing the expanded polyglutamine (HTTQ94) of mHTT showed that it is able to promote both the ACSL4-dependent and the ACSL4-independent ferroptosis. Surprisingly, inactivation of the ACSLA-dependent ferroptosis has no obvious effect on the life span of Huntington's disease mouse. Moreover, by using RNAi-mediated screening, ALOX5 was identified as a major factor required for the ACSLA-independent ferroptosis induced by HTTQ94. Although ALOX5 is not required for the ferroptotic responses triggered by common ferroptosis inducers such as erastin, loss of ALOX5 expression abolishes HTTQ94-mediated ferroptosis upon reactive oxygen species (ROS)-induced stress. Interestingly, ALOX5 is also required for HTTQ94-mediated ferroptosis in neuronal cells upon high levels of glutamate. Mechanistically, HTTQ94 activates ALOX5-mediated ferroptosis by stabilizing FLAP, an essential cofactor of ALOX5-mediated lipoxygenase activity. Notably, inactivation of the Alox5 gene abrogates the ferroptosis activity in the striatal neurons from the HD mice; more importantly, loss of ALOX5 significantly ameliorates the pathological phenotypes and extends the life spans of these HD mice. Taken together, these results demonstrate that ALOX5 is critical for mHTT-mediated ferroptosis and suggest that ALOX5 is a potential new target for Huntington's disease.

Huntington's disease (HD) is an autosomal dominant hereditary neurodegenerative disease characterized by progressive cognitive, behavioral, motor dysfunctions and short life-spans (Bates et al. 2015). HD is caused by a CAG trinucleotide repeat expansion in Exon 1 of the HTT gene, which results in an expanded polyglutamine (polyQ) tract in the encoded huntingtin protein, referred to as mutant huntingtin (mHTT). The mHTT exhibits toxic gain of functions causing neuronal dysfunction and cell death (MacDonald et al. 1993; Bates 2003). The proteolytic cleavage of mHTT is a key event in the molecular pathogenesis of HD and it is believed that the N-terminal fragment containing the expanded polyglutamine of mHTT plays an important role in the pathogenesis of HD (Martindale et al. 1998; Lunkes et al. 2002; Graham et al. 2006). Although the genetic cause of HD is well established, the cellular and molecular mechanisms involved in mHTT-mediated early neuronal dysfunction and late neurodegeneration are not fully understood.

Redox signaling is essential for normal brain function, being involved in memory consolidation, neuronal differentiation, and plasticity. Ferroptosis is a regulated form of non-apoptotic cell death driven by excess accumulation of lipid peroxidates critically regulated by the redox signaling (Stockwell et al. 2020). Accumulating evidence indicates that ferroptosis may be involved in both animal models and human patients with Huntington's disease. For example, in transgenic HD mouse models and patients, most of neuronal death did not exhibit the classic apoptotic features (Turmaine et al. 2000; Hickey and Chesselet 2003). Moreover, higher levels of lipid peroxidation are observed as a principal characteristic in HD patients (Klepac et al. 2007). It was reported that increased levels of lipid peroxidation were detected in corticostriatal brain slices (Skouta et al. 2014) and were colocalized with mHTT inclusions in the striatal neurons (Lee et al. 2011). Inhibition of lipid peroxidation with ferrostatin-1(Ferr-1) significantly rescued the cell death in cellular models of Huntington's disease (HD) (Skouta et al. 2014). On the other hand, lower GSH levels are another characteristics in HD patients (Klepac et al. 2007). Indeed, decreased GSH and GSH-S-transferase were detected in the striatum, cortex and hippocampus in 3-nitropropionic acid (3-NP)-induced HD mouse (Kumar et al. 2010). mHTT can directly interact with mitochondrial proteins, such as translocase of the inner membrane 23 (TIM23), disrupting mitochondrial proteostasis and favoring ROS production and HD progression (Yablonska et al. 2019). Thus, it is very important to examine whether mHTT is directly involved in regulating ferroptosis and more importantly, the molecular factors that mediate mHTT-dependent ferroptosis need to be delineated.

Ferroptosis, an iron-dependent form of non-apoptotic cell death driven by lipid-based reactive oxygen species (ROS), is tightly linked with human diseases including neurodegenerative diseases (Stockwell, 2020). Lipid peroxides can be eliminated by glutathione peroxidase 4 (GPX4) and its co-factor glutathione (GSH) (Stockwell et al., 2017). Thus, ferroptosis is commonly induced by pharmacological agents that disrupt this lipid repair system, that directly or indirectly lead to GPX4 inhibition (Stockwell et al., 2017). Notably, by using genome-wide haploid and CRISPR-Cas9-based screening, acyl-CoA synthetase long-chain family member 4 (ACSL4) was identified as an essential factor for ferroptosis induced by GPX4 inhibitors (Doll et al. 2017; Kagan et al. 2017). The critical role of ACSL4 in ferroptosis relies on its ability to incorporate arachidonic acid and adrenic acid into phosphatidylethanolamines and thereby provides the main substrates for peroxidation (Kagan et al. 2017). In addition to GPX4-mediated neutralization of lipid peroxidation, the levels of cellular lipid peroxides can be induced enzymatically by the lipoxygenases. Indeed, recent studies showed that the ferroptotic response induced by the ALOX12 lipoxygenase plays an important role in catalyzing lipid peroxidation and p53-mediated effect on Myc-induced lymphomagenesis (Chu et al., 2019). Together, these studies indicate that two types of ferroptosis are critical for oxidative stress responses: ACSL4-dependent ferroptosis is induced by these common ferroptosis inducers mainly including GPX4 inhibitors whereas ACSL4-independent ferroptosis is induced by high levels of reactive oxygen species (ROS).

Here, expression of the mHTT fragment with the expanded 94 glutamine residues (HTTQ94) showed that it directly promotes ferroptosis in both neuronal cells and neuroblastoma cells upon erastin treatment. As expected, inhibition of ACSL4 in those cells completely abrogated the ferroptotic response induced by HTTQ94; surprisingly, however, inactivation of the acsl4 gene failed to show any significant effect in the Huntington's disease transgenic mouse model (HD-N171-82Q). Moreover, ALOX5 was identified as a major mediator for HTTQ94-driven ferroptosis upon ROS-induced stress. Interestingly, neither ACSL4 nor GPX4 is required for ALOX5-dependent ferroptosis induced by HTTQ94. Moreover, knockout of the Alox5 gene abrogated ferroptosis in the striatal neurons from the HD mice and significantly improved the pathological phenotypes of Huntington's disease mice. Thus, the data described herein reveals a novel ferroptosis pathway critically involved in the pathological mechanism of Huntington's disease.

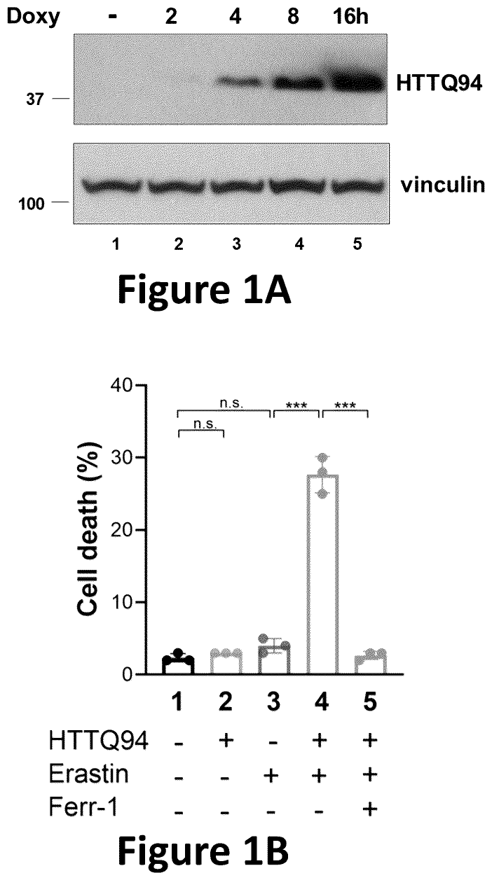

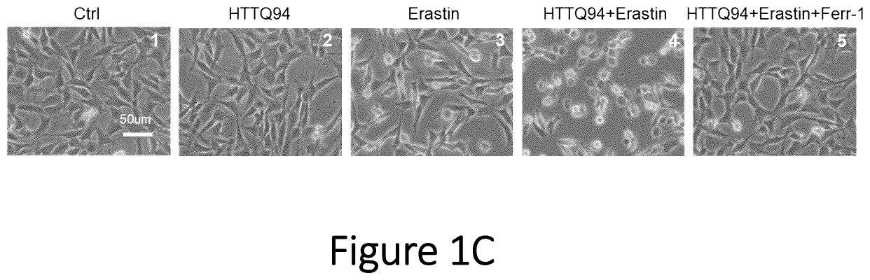

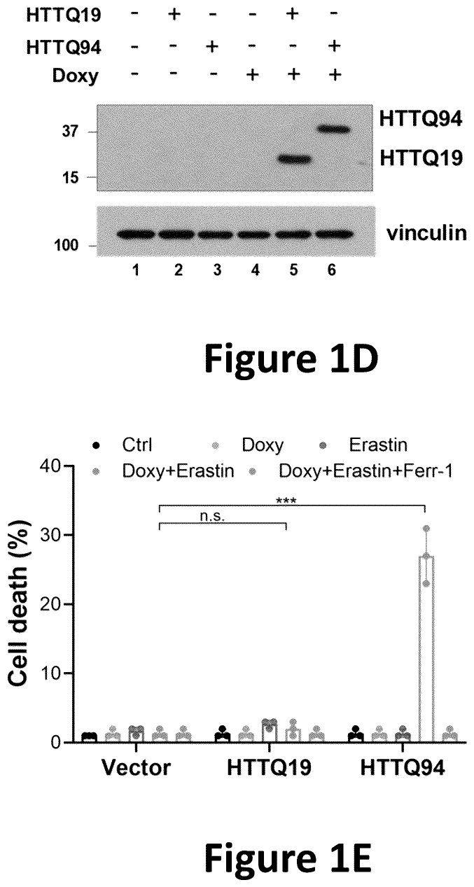

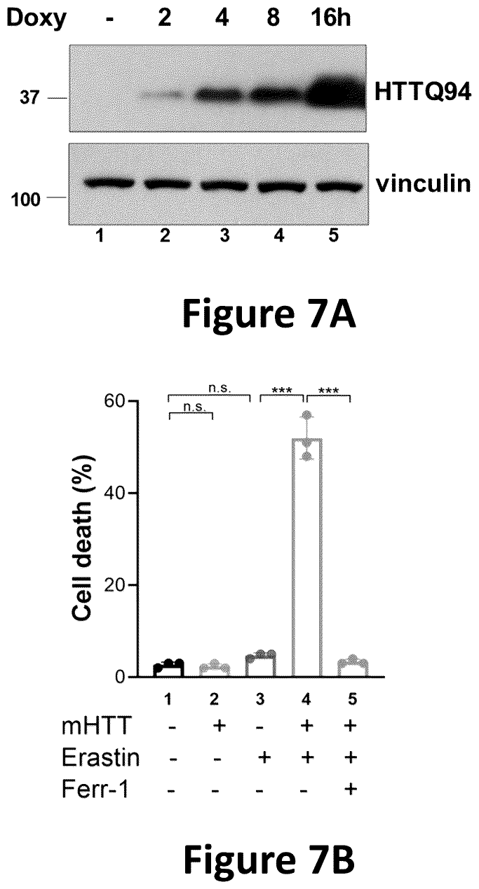

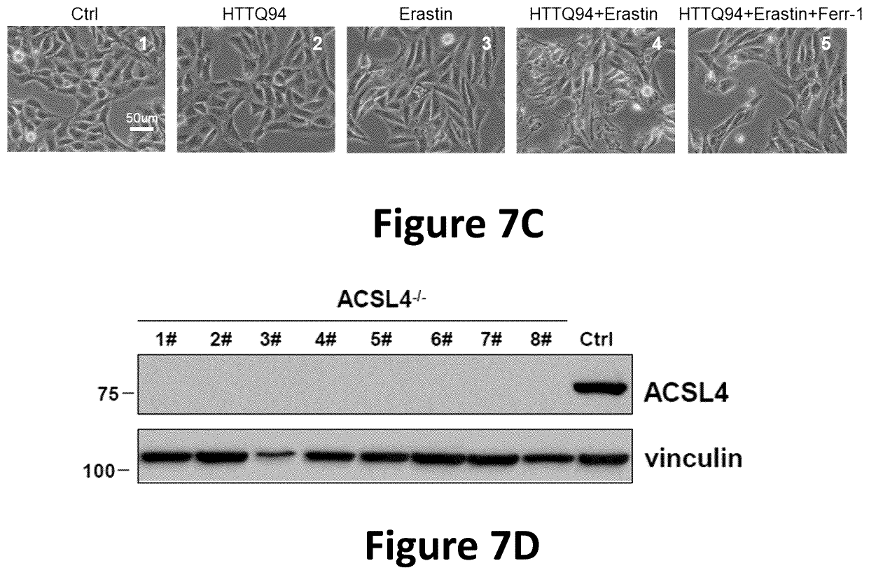

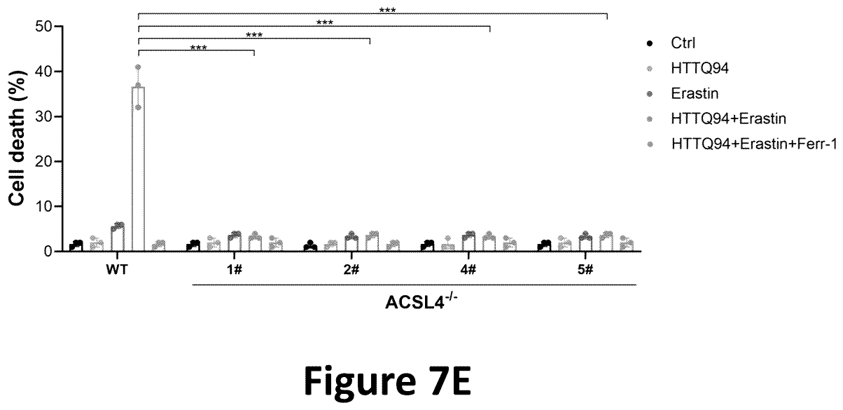

To elucidate the potential role of ferroptosis in Huntington's disease, whether mHTT is directly involved in regulating ferroptotic responses was first examined. To this end, the mouse hippocampal neuronal cell line HT-22 was used to establish HTTQ94 tet-on inducible cell lines, in which expression of the N-terminal fragment mutant huntingtin protein containing the expanded 94 glutamine residues (HTTQ94) can be induced by doxycycline. As shown in FIG. 1A, high levels of HTTQ94 were indeed induced upon the treatment of doxycycline in those cells. Notably, although induction of HTTQ94 alone did not significantly induce any cell death (FIG. 1B,C), the combination of HTTQ94 induction and the erastin treatment produced high levels of cell death than either treatment alone and the cell death was completely inhibited by ferrostatin-1, a specific ferroptosis inhibitor (FIG. 1B,C). To corroborate these findings, the similar assays were performed by establishing a human neuroblastoma SK-N-BE(2)C HTTQ94 inducible cell line (FIG. 7A). Notably, although native SK-N-BE(2)C cells are highly resistant to the erastin treatment, high levels of ferroptosis were induced upon HTTQ94 induction in those cells (FIG. 7B, C). Moreover, a control tet-on inducible cell line, in which expression of the N-terminal fragment wild-type huntingtin protein containing the normal 19 glutamine residues (HTTQ19) was established (FIG. 1D). Indeed, in contrast to the effects induced by HTTQ94 expression, expression of HTTQ19 failed to sensitize the cells to ferroptosis (FIG. 1E). Taken together, these data demonstrate that HTTQ94 is directly involved in promoting ferroptosis in neuronal cells as well as other cell types.

### Example 2: Inactivation of ACSL4 has No Obvious Effect on the Pathological Phenotypes and the Life Span of Huntington's Disease Mouse

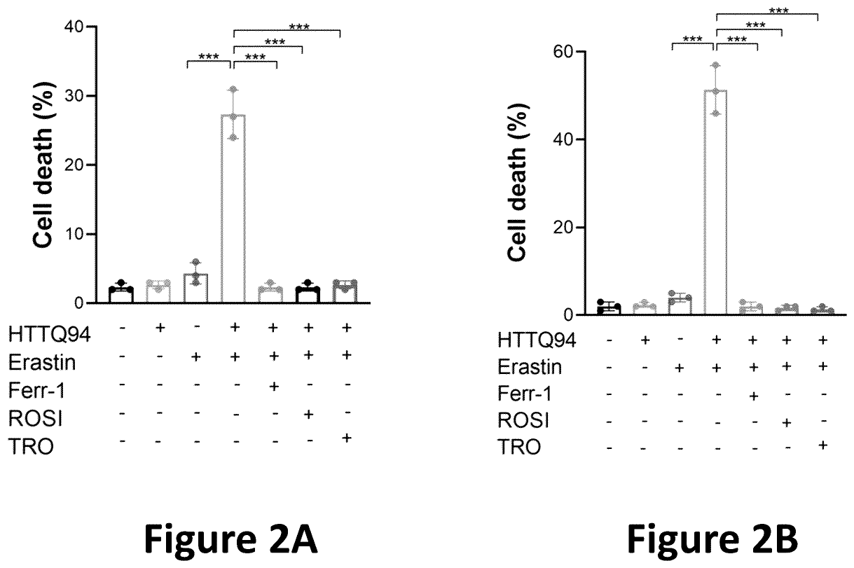

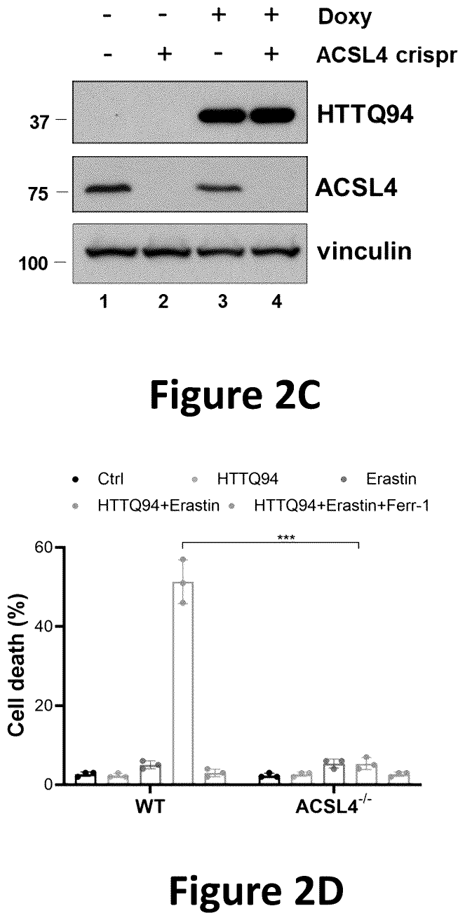

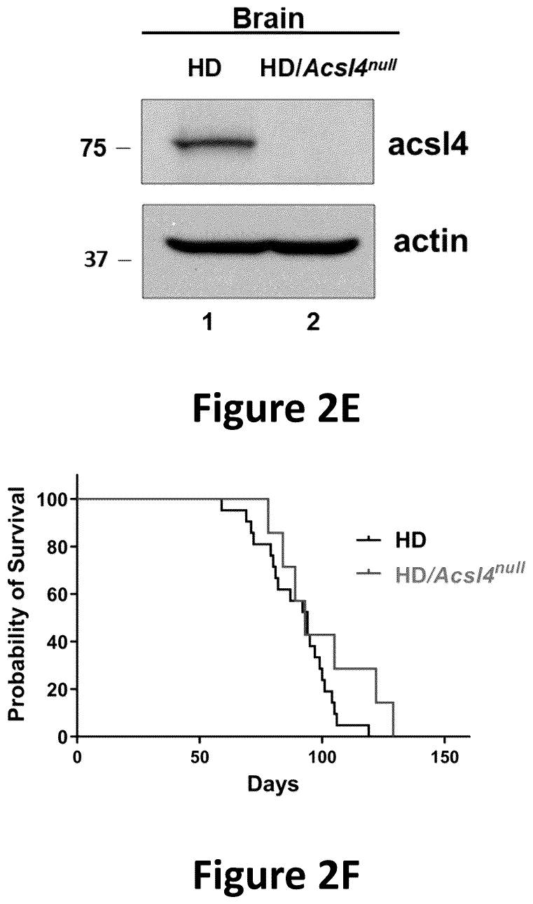

Recent studies indicate that acyl-CoA synthetase long-chain family member 4 (ACSL4) is essential for the ferroptotic responses induced by either erastin or GPX4 inhibitors (Doll et al. 2017; Kagan et al. 2017; Stockwell et al. 2017). To dissect the molecular mechanism of mHTT mediated ferroptosis, the function of the ACSLA inhibitors in HTTQ94-mediated ferroptosis was first examined. Both the ACSL4 inhibitors, rosiglitazone (ROSI) and troglitazone (TRO), prevented HTTQ94 mediated ferroptosis in both HT-22 and SK-N-BE(2)C cells (FIG. 2A, B). To further validate the role of ACSLA in HTTQ94-mediated ferroptosis, ACSL4 knockout cells from the HTTQ94 tet-on SK-N-BE(2)C cell line by using CRISPR/cas9 method was generated. Western blot analysis revealed that ACSL4 protein was indeed undetectable in ACSL4-Crispr cells although the same levels of HTTQ94 were induced in both control and ACSL4-Crispr cells (FIG. 2C). Indeed, the ferroptosis response induced by HTTQ94 was completely abrogated in ACSL4-null cells under the same conditions (FIG. 2D). The results were further confirmed by four independent ACSL4 knockout cell lines under the same conditions (FIG. 7D, E). These data demonstrate that HTTQ94 plays an important role in promoting the ferroptotic response upon GPX4 inhibition and that this activity can be completely abolished upon loss of ACSL4.

By establishing Acsl4 knockout mice, the GPX4-dependent ferroptosis can also be inactivated upon loss of ACSL4 expression in vivo (Chu et al. 2019). To further elucidate the role of mHTT mediated ferroptosis in pathogenesis of Huntington's disease, whether inactivation of the GPX4 dependent ferroptosis has any effect on the phenotypes observed in Huntington's disease mouse models was examined. To this end, a well-established HD transgenic mouse model (HD-N171-82Q), in which a HTTQ82 protein with the N-terminal fragment (171 amino acids) and an 82 residue glutamine repeat was constitutively expressed (Schilling et al. 1999) was used. These mice developed behavioral abnormalities, including loss of coordination, tremors, hypokinesis and abnormal gait and died prematurely within ˜120 days after birth (Schilling et al. 1999). After crossed these transgenic HD mice with Acsl4 knockout mice, the HD/Acsl4-null mice was successfully generated. As expected, the acsl4 protein was undetectable in the brain of the HD/Acsl4-null mice (FIG. 2E). However, no obvious difference regrading behavioral abnormalities was observed between the HD mice and the HD/Acsl4-null mice. More importantly, there was no statistically significant difference of the median survival comparing the HD/Acsl4-null mice with the control HD mice (FIG. 2F), suggesting loss of acsl4 does not affect the life span of HD mice. Of note, these HD mice showed abnormalities very early (about 2 month old) (Schilling et al. 1999). Moreover, it is very likely that loss of Acs14 may also cause (partial) embryonic lethality (Chu et al., 2019), which made this breeding program very difficult, causing the difference in the sample sizes (HD mice (n=21) and HD/Ascl4-null mice (n=7)). Future studies are required to examine whether other factors may contribute to this phenotype. Thus, although ACSL4 is essential for HTTQ94-mediated ferroptosis induced by GPX4 inhibition, this pathway was not major effects on the life span of Huntington's disease mice.

### Example 3: HTTQ94 is Able to Induce the ACSL4-Independent Ferroptosis Upon ROS-Induced Stress

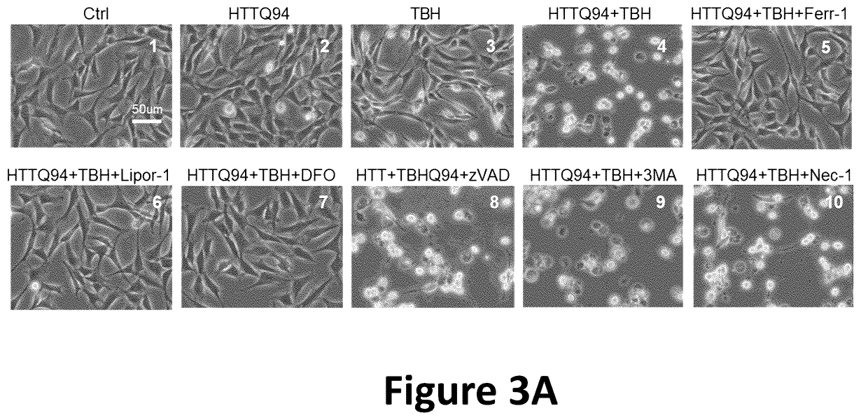

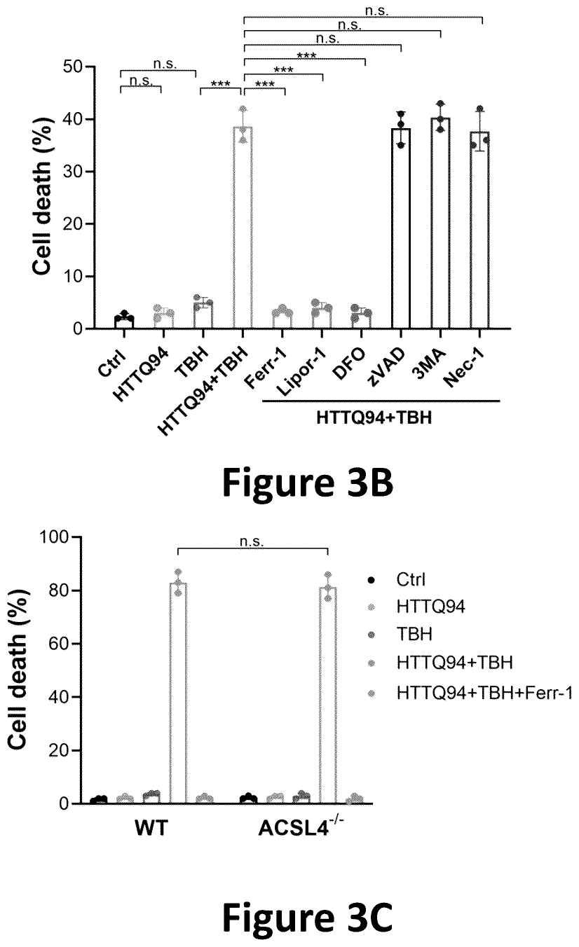

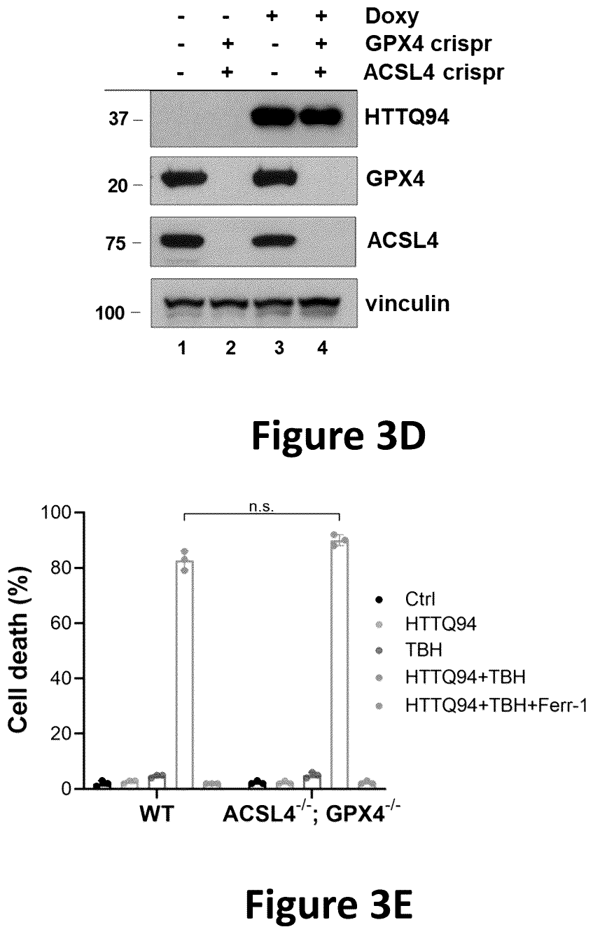

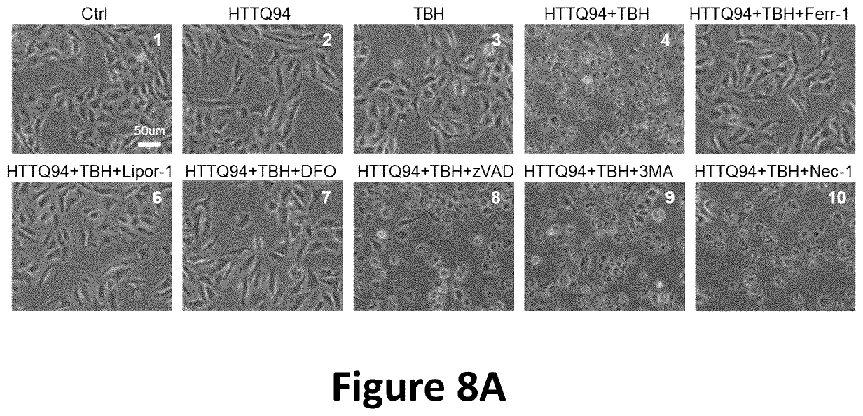

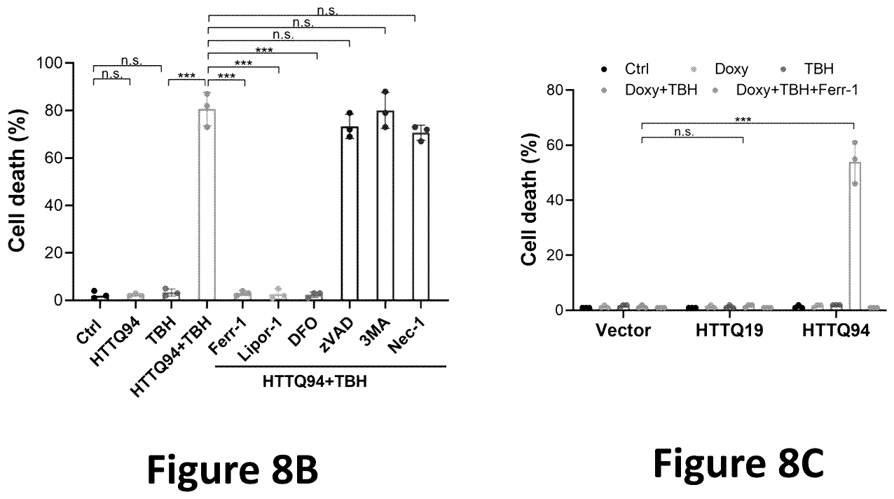

The previous study showed that the ferroptotic response can also be induced upon ROS stress, independent of the ACSL4 status (Chu et al. 2019). Since numerous studies indicate the importance of ROS stress in Huntington's disease pathogenesis (Paul and Snyder 2019), whether HTTQ94 promotes ROS-induced ferroptosis in the established HT-22 HTTQ94 inducible cell line upon ROS-induced stress, generated by tert-butyl hydroperoxide (TBH) was first examined. As shown in FIG. 3A and FIG. 3B, although HTTQ94 induction, or TBH treatment alone failed to induce any cell death, high levels of cell death were induced upon the combination of HTTQ94 expression and ROS stress. Moreover, these HTTQ94-mediated responses were specifically blocked by several well-known ferroptosis inhibitors (e.g., Ferr-1, Lipro-1 and DFO; FIG. 3A, B) but not by the inhibitors of other cell death pathways, such as apoptosis, autophagy or necroptosis (FIG. 3A, B). Similar results were also obtained in the SK-N-BE(2)C HTTQ94 inducible cell line upon ROS-induced stress (FIG. 8A, B). Moreover, in contrast to the effects induced by HTTQ94 expression, no obvious ferroptotic cell death was induced upon expression of the N-terminal fragment wild-type huntingtin protein containing the normal 19 glutamine residues (HTTQ19) under the same treatment (FIG. 8C).

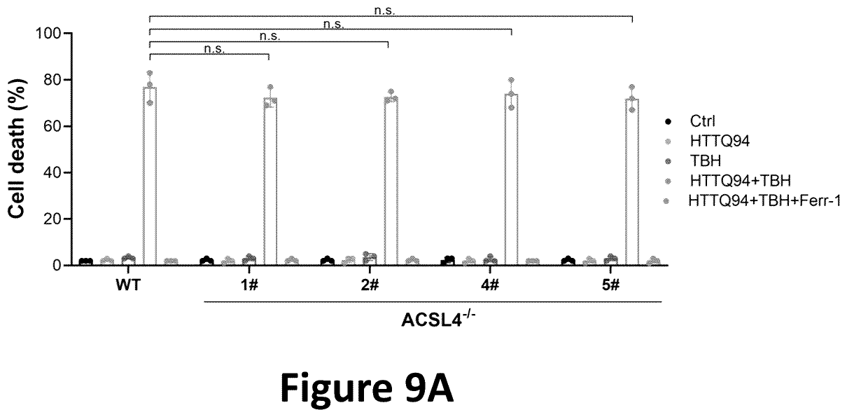

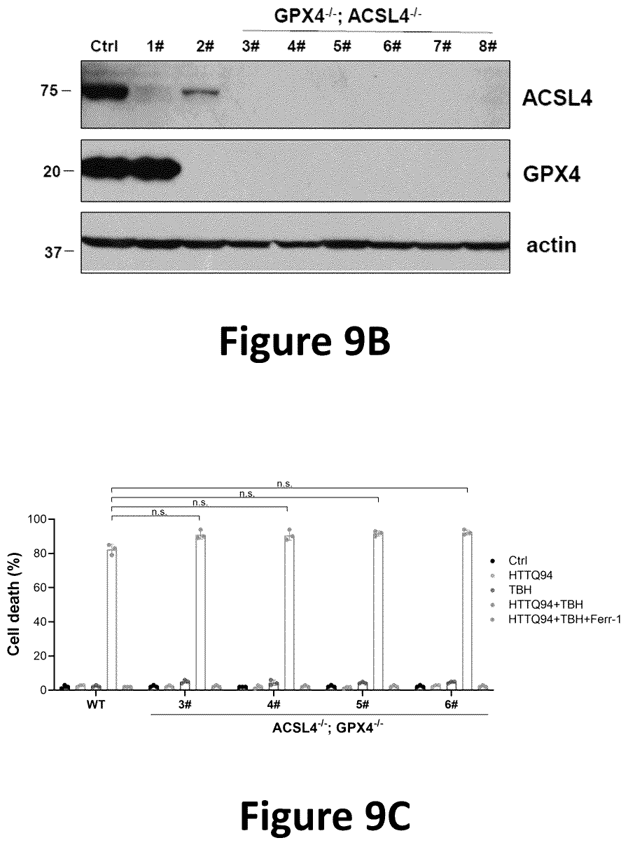

Next, whether this type of ferroptosis is dependent on ACSL4 by using the established ACSL4 knockout cell lines was examined. Indeed, the ferroptosis response induced by HTTQ94 remained intact in ACSL4-null cells under the same conditions (FIG. 3C). The similar results were observed in four independent ACSL4 knockout cell lines (FIG. 9A). Since ACSL4 is essential for ferroptosis induced by GPX4 inhibition, it is likely that HTTQ94-mediated ferroptosis upon ROS-induced stress is independent of GPX4 function. However, this notion cannot be directly tested since GPX4-null cells do not survive under normal conditions unless ACSL4 is co-deleted. To address whether HTTQ94-mediated ferroptosis can be induced independent of GPX4 function, ACSL4/GPX4 double-knockout derivatives of the SK-N-BE(2)C HTTQ94 inducible cell line was generated. As shown in FIG. 3D, neither ACSL4 nor GPX4 was detectable in these ACSL4/GPX4-null cells with inducible HTTQ94 expression. Nevertheless, the ferroptotic cell death was readily induced upon HTTQ94 expression in ACSL4/GPX4-null cells under ROS stress conditions (FIG. 3E). The result was again confirmed by using several independent ACSL4/GPX4 knockout cell lines under the same conditions (FIG. 9B, C). Taken together, these data demonstrate that HTTQ94 is able to induce ferroptosis under ROS stress conditions independent of either ACSL4 or GPX4.

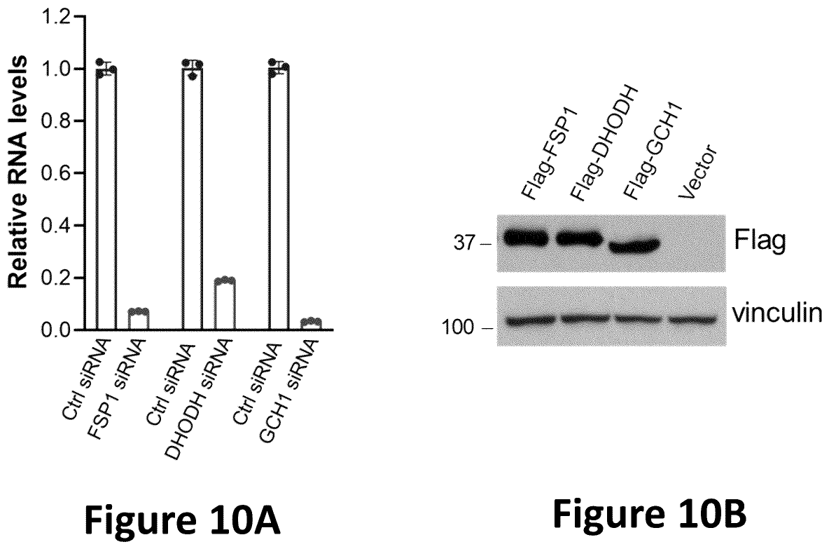

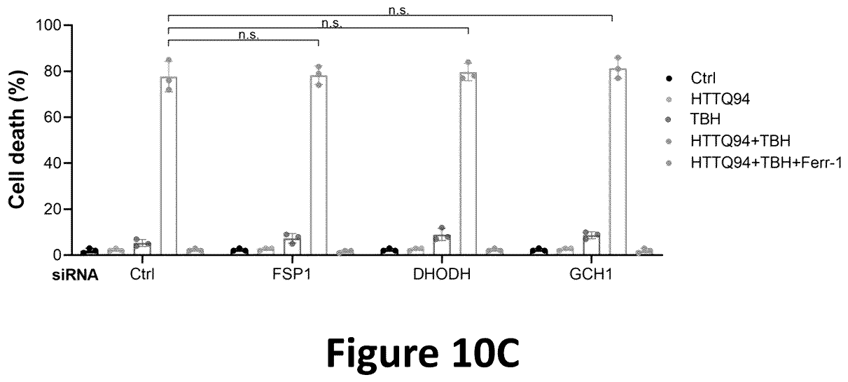

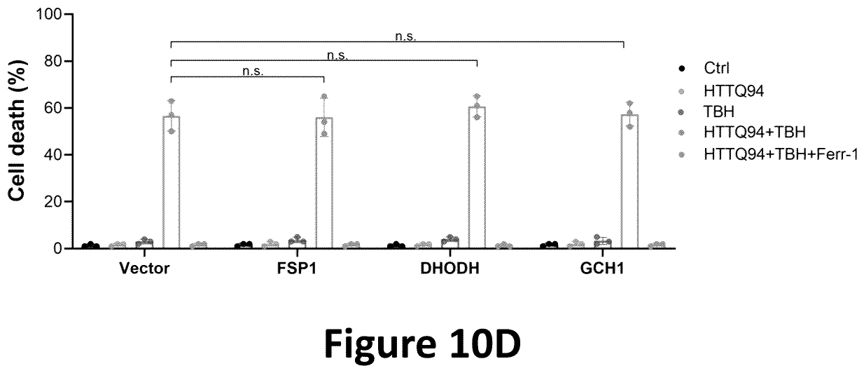

Finally, in addition to GPX4, several important cellular factors have been identified critically involved in regulating ferroptotic responses through completely different mechanisms such as FSP1, GCH1 and DHODH (Doll et al. 2019; Bersuker et al. 2019; Kraft et al. 2020; Mao et al. 2021). To examine whether these ferroptosis regulators play a role in HTTQ94-mediated ferroptosis upon ROS stress, RNAi-mediated depletion in the tet-on HTTQ94-inducible SK-N-BE(2)C cells (FIG. 10A) and overexpression in the tet-on HTTQ94-inducible H1299 cells (FIG. 10B) were examined to test whether HTTQ94-mediated ferroptosis is affected by depletion of FSP1, GCH1 or DHODH. As shown in FIG. 10C and FIG. 10D, HTTQ94-mediated ferroptotic responses upon ROS stress remained intact upon either depletion or overexpression of FSP1, GCH1 and DHODH, respectively. Together, these data suggest that HTTQ94-mediated cell death upon high levels of ROS may represent a new ferroptosis pathway that needed to be further elucidation.

### Example 4: ALOX5 is Required for HTTQ94 Mediated Ferroptosis Upon ROS-Induced Stress

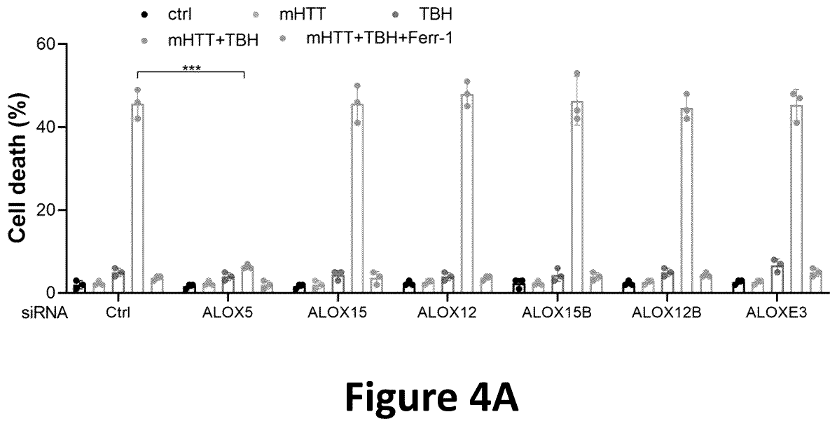

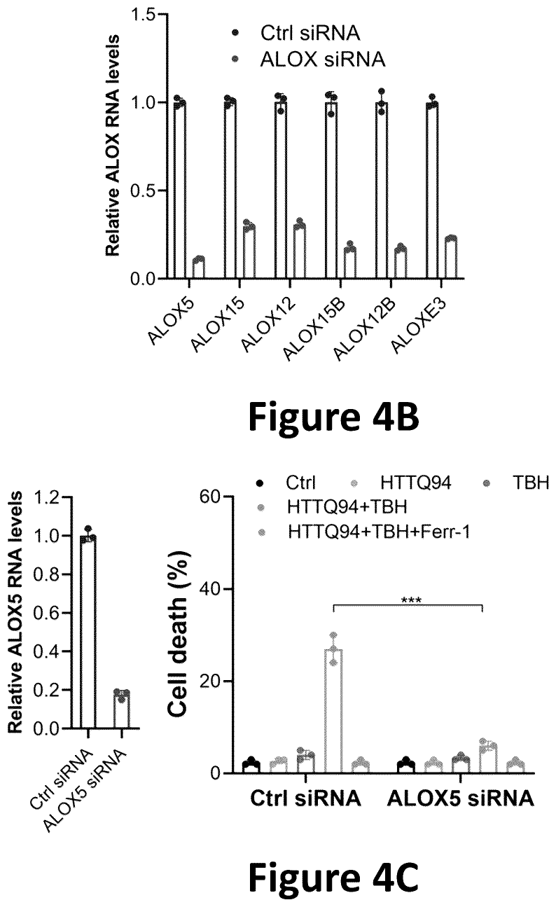

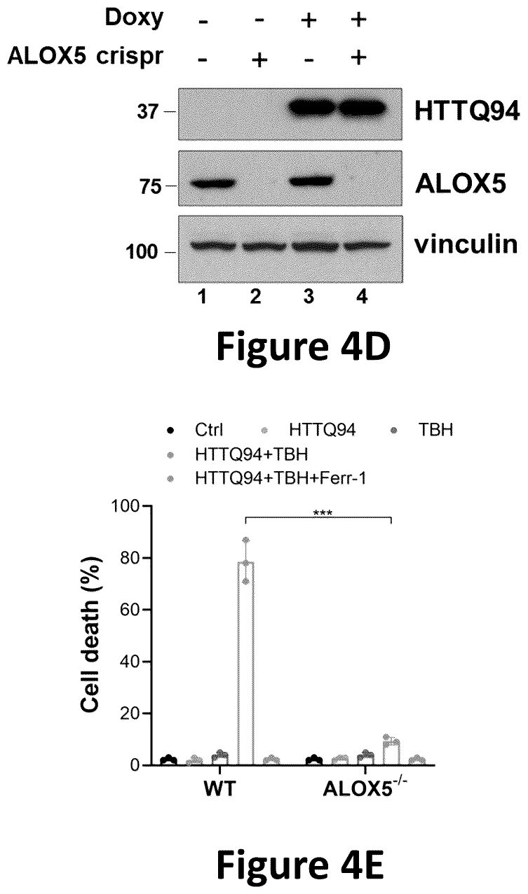

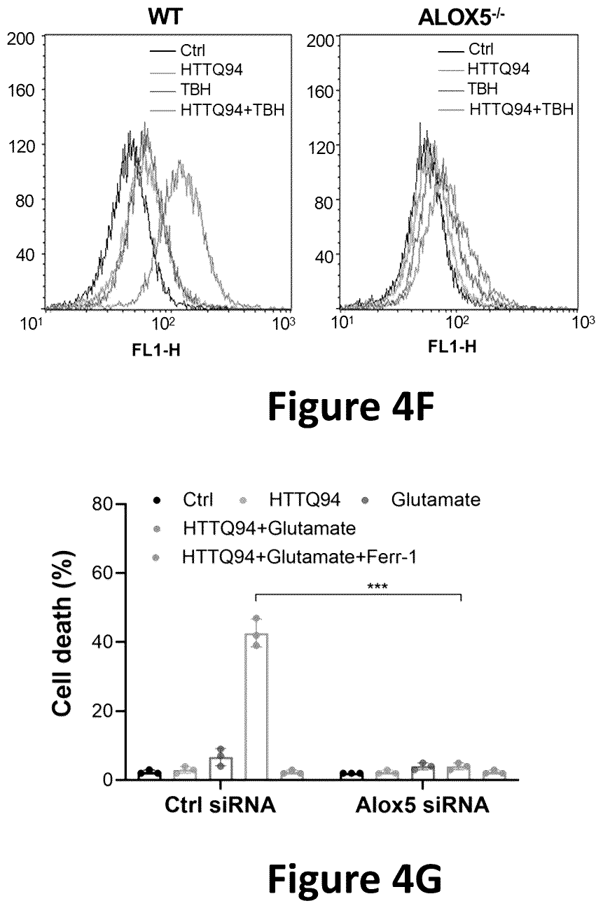

Notably, the recent studies showed that ferroptosis can be induced by activation of the lipoxygenases independent of these known factors (Chu et al. 2019). The mammalian lipoxygenase family consists of six isoforms (ALOXE3, ALOX5, ALOX12, ALOX12B, ALOX15, and ALOX15B) with differing substrate specificities (Mashima and Okuyama 2015). To examine whether any of these lipoxygenases is required for mHTT-mediated ferroptosis upon ROS stress, an RNAi-mediated loss-of-function screen was performed to test whether depletion of individual isoforms affects HTTQ94 dependent ferroptosis. To this end, the tet-on HTTQ94-inducible SK-N-BE(2)C cells were transfected with siRNAs (Dharmacon SMART-pools) specific for each of the six lipoxygenases and treated with TBH to induce ferroptosis. Significantly, mHTTQ94-mediated ferroptosis upon ROS stress was markedly blocked by RNAi-mediated depletion of ALOX5, but not by the depletion of the other five lipoxygenases (FIG. 4A). Quantitative polymerase chain reaction (qPCR) analyses confirmed that the expression of each of the six lipoxygenase isoforms was individually abrogated by RNAi-mediated depletion (FIG. 4B). To corroborate this finding, RNAi-mediated depletion of ALOX5 in the HTTQ94 tet-on HT-22 cells was performed (FIG. 4C). Indeed, HTTQ94-mediated ferroptosis upon ROS stress was abolished upon loss of ALOX5 expression (FIG. 4C).

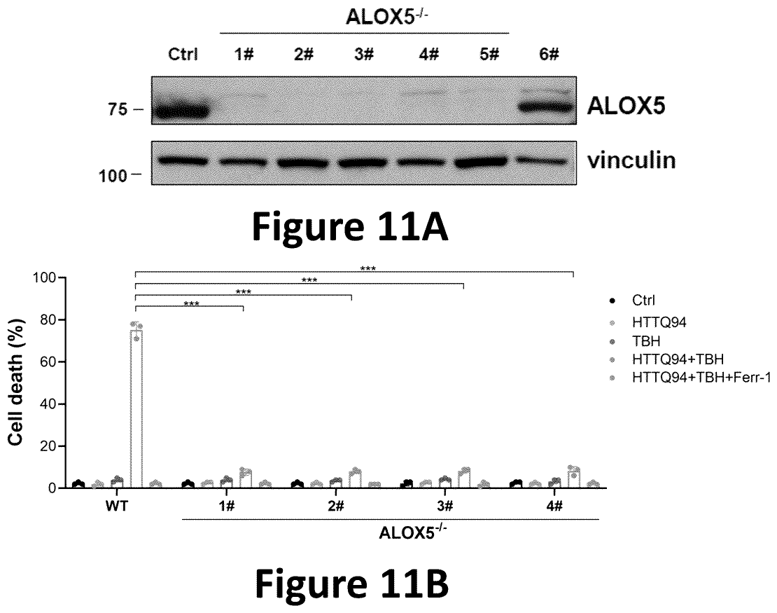

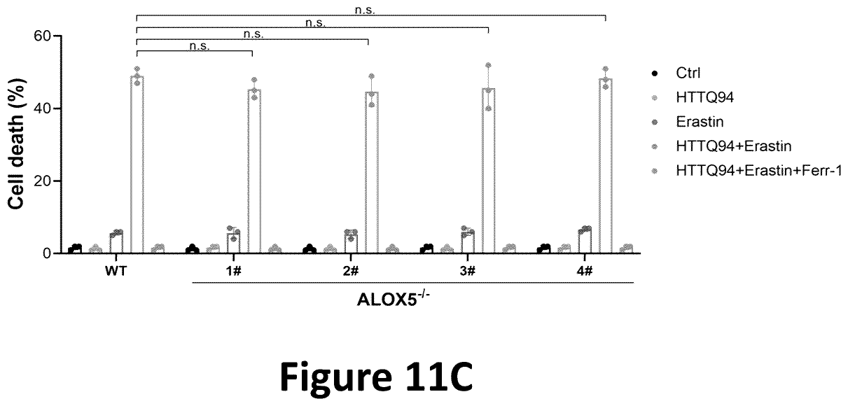

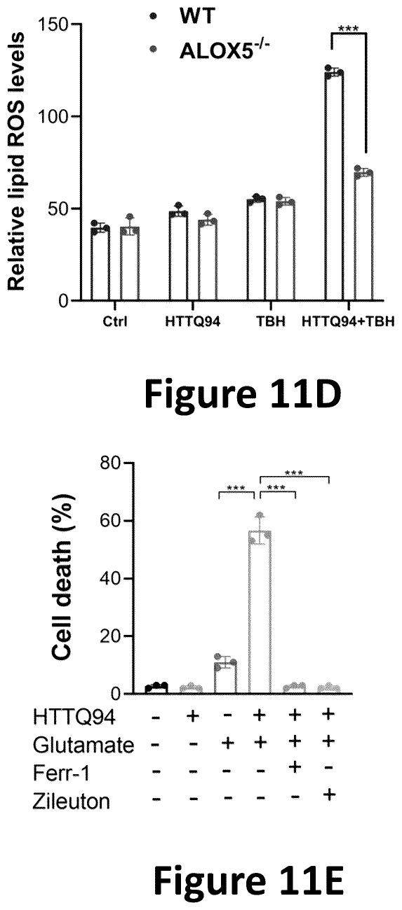

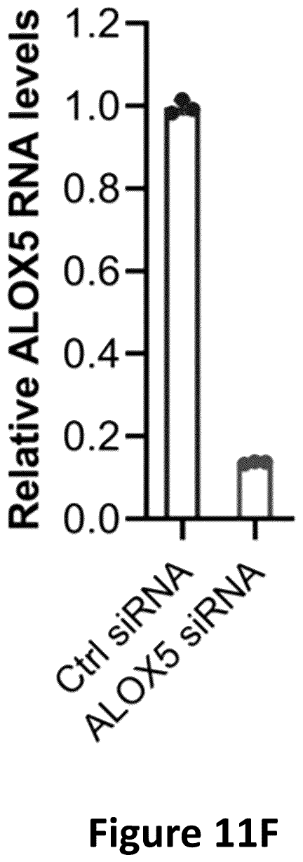

To further validate the role of ALOX5 in HTTQ94-mediated ferroptosis, ALOX5 knockout cells by CRISPR/cas9 technology was generated by using the HTTQ94 tet-on SK—N-BE(2)C cells (FIG. 11A). As shown in FIG. 4D, the ALOX5 protein was undetectable in ALOX5 null cells and loss of ALOX5 expression dramatically abolished the high levels of HTTQ94-mediated ferroptosis upon ROS stress (FIG. 4E). The results were further confirmed by four independent ALOX5 knockout cell lines under the same conditions (FIG. 11B) although the ferroptosis response induced by HTTQ94 remained intact in ALOX5-null cells under erastin treatment (FIG. 11C). Moreover, by using flow cytometry analysis with C11-BODIPY staining, the levels of endogenous membrane lipid peroxidation, a key marker of ferroptosis, were significantly induced upon HTTQ94 expression but these effects were largely abrogated upon loss of ALOX5 (FIG. 4F; FIG. 11D). Taken together, these data demonstrate that ALOX5 is essential for HTTQ94 mediated ferroptosis upon ROS-induced stress by increasing the lipid peroxidation levels.

Glutamate, the most abundant endogenous excitatory neurotransmitter in brain, plays a crucial role in neuronal tissue damage. Recently, several studies showed that ferroptosis is regulated by glutamate (Liu et al. 2015; Maher et al. 2020; Xie et al. 2022). The HTTQ94 inducible HT-22 cell line was treated with glutamate to investigate whether HTTQ94 also promotes glutamate induced ferroptosis. Indeed, high levels of cell death were induced upon HTTQ94 expression in the presence of glutamate (FIG. 11E), which was inhibited by ferrostatin-1, indicating that glutamate-induced ferroptosis is significantly enhanced by HTTQ94 expression. More importantly, HTTQ94-mediated ferroptosis upon glutamate induction could be completely blocked by Zileuton (Liu et al. 2015), a well-known inhibitor of ALOX5 (FIG. 11E). Next, RNAi-mediated knockdown of ALOX5 (FIG. 11F) was performed to further examine the role of ALOX5 in regulating glutamate-mediated ferroptosis. As shown in FIG. 4G, HTTQ94-mediated ferroptosis was indeed abrogated in ALOX5 knockdown cells. Thus, these data indicate that ALOX5 is also critical for glutamate-mediated ferroptosis upon HTTQ94 expression.

### Example 5: Mechanistic Insight into ALOX5 Activation Induced by HTTQ94 Expression

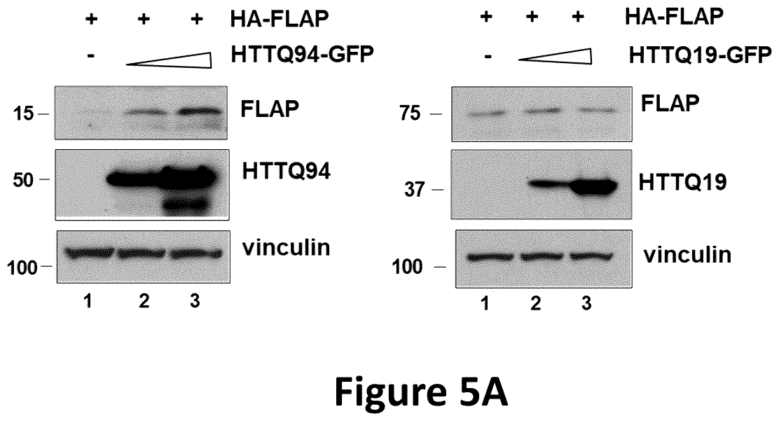

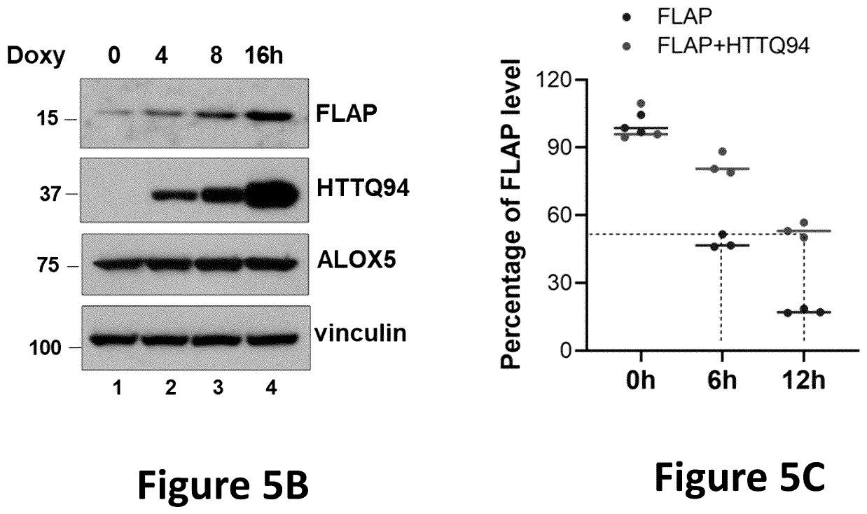

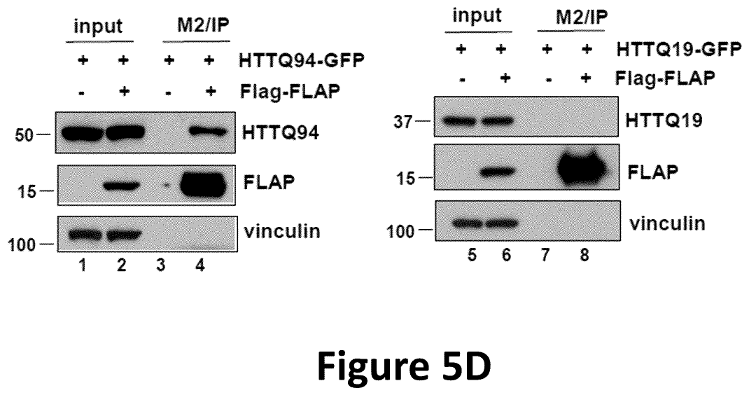

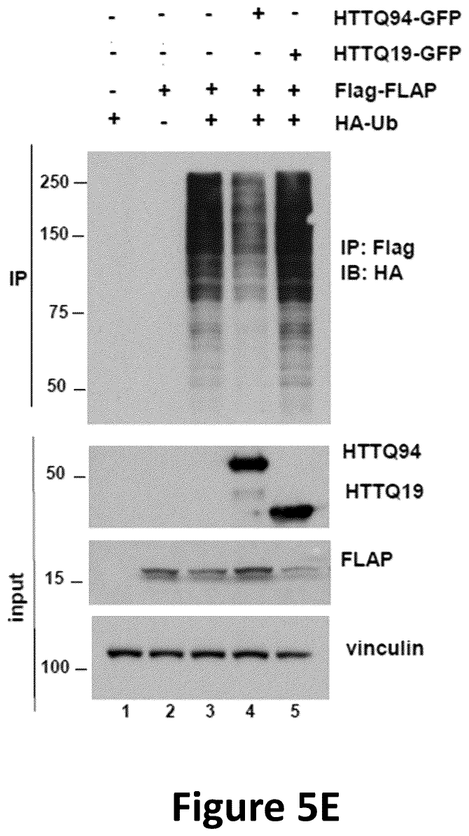

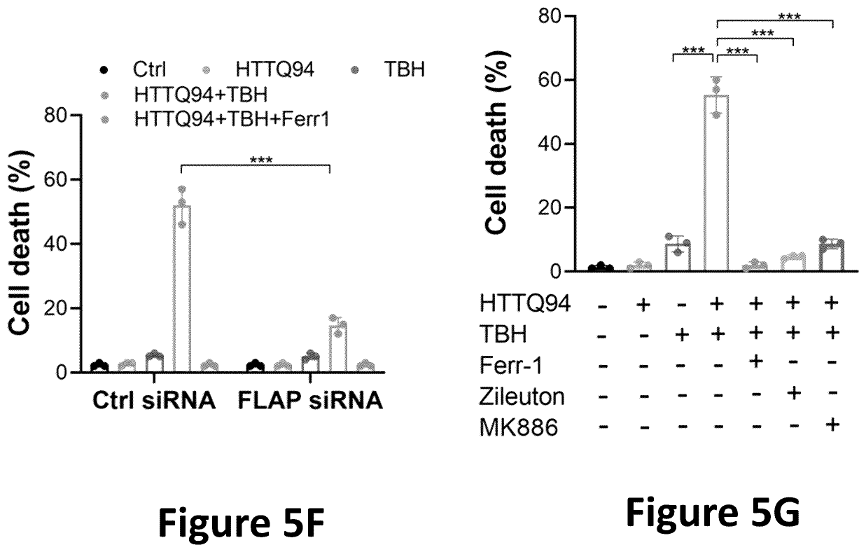

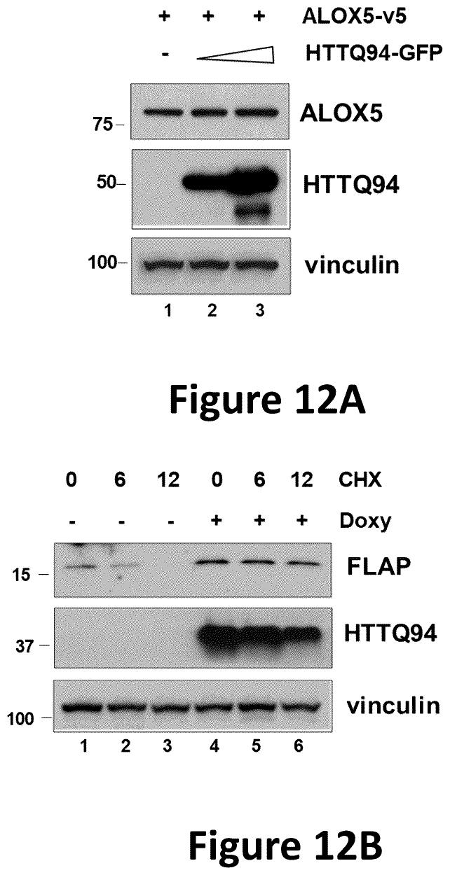

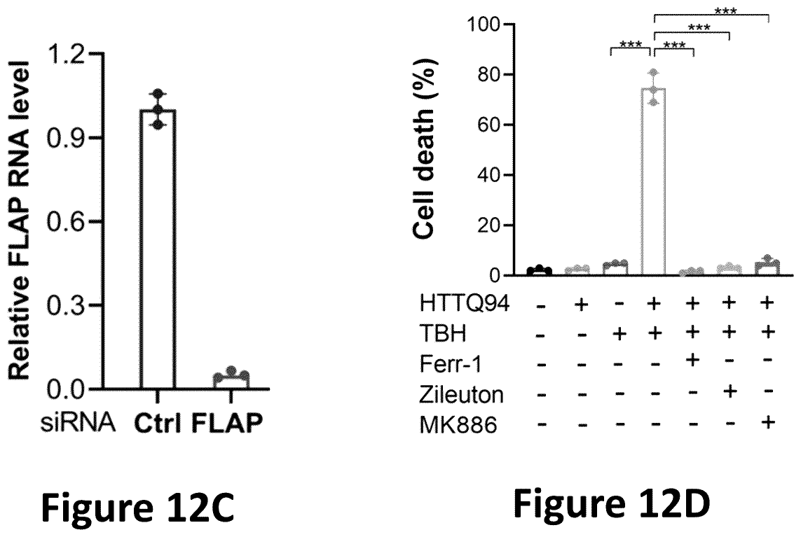

To dissect the mechanism by which HTTQ94 promotes ALOX5-dependent ferroptosis, whether the protein levels of ALOX5 is regulated by HTTQ94 expression was tested. As shown in FIG. 12A, western blot analysis revealed that HTTQ94 overexpression had no obvious effect on the levels of ALOX5 protein, suggesting that ALOX5 protein stability is not the key factor in HTTQ94-mediated regulation. Like ALOX12, ALOX5 is a member of the lipoxygenase family containing intrinsic lipoxygenase activity (Chu et al. 2019). However, unlike other lipoxygenases, ALOX5 requires a specific cofactor called FLAP (the 5-lipoxygenase-activating protein) for catalyzing the lipid oxygenation reaction (Peters-Golden and Brock 2003; Mashima and Okuyama 2015). Next, whether the protein stability of FLAP can be regulated by HTTQ94 was examined. Notably, the steady-state levels of FLAP were markedly increased upon HTTQ94 co-expression (FIG. 5A), but were not affected by co-expression of the N-terminal fragment wild-type huntingtin protein containing the normal 19 glutamine residues (HTTQ19) (FIG. 5A). Moreover, the levels of endogenous FLAP, but not ALOX5, were increased in a dosage-dependent manner upon HTTQ94 induction (FIG. 5B); the half-life of endogenous FLAP was significantly extended upon HTTQ94 expression from 6 hours to more than 12 hours (FIG. 5C; FIG. 12B). These data indicate that HTTQ94 but not the wild-type counterpart (HTTQ19) is able to induce the protein stabilization of FLAP.

Next, the interaction between HTTQ94 (or HTTQ19) and FLAP was examined in human cells. To this end, cells were transfected with a HTTQ94 (or HTTQ19) expression vector in the presence or absence of a vector encoding Flag-FLAP in 293 cells. As shown in FIG. 5D, HTTQ94 was readily detected in the immunoprecipitated complexes of Flag-FLAP; however, the wild-type counterpart (HTTQ19) failed to show up in the immunoprecipitated complexes of Flag-FLAP under the same conditions (FIG. 5D), suggesting that HTTQ94 specifically interacts with FLAP. To further dissect the mechanism by which HTTQ94 promotes FLAP stabilization, the effect on the levels of FLAP ubiquitination by HTTQ94 was tested in cells. Indeed, the levels of ubiquitinated FLAP were dramatically reduced upon expression of HTTQ94 but not HTTQ19 (FIG. 5E), suggesting that HTTQ94 stabilizes FLAP by suppressing FLAP ubiquitination. Finally, to evaluate the importance of FLAP in HTTQ94-mediated effect, whether HTTQ94-mediated ferroptosis also requires FLAP was tested. To this end, the endogenous FLAP was knocked down by RNAi in the HTTQ94 inducible cells and then the effects on HTTQ94-mediated ferroptosis was tested. Indeed, upon RNAi-mediated depletion of FLAP (FIG. 12C), HTTQ94-mediated ferroptosis upon ROS stress was largely abrogated (FIG. 5F). Moreover, whether pharmacological inhibition of FLAP or ALOX5 with specific inhibitors of FLAP or ALOX5 (Pergola et al. 2014; Liu et al. 2015) has any effect on HTTQ94-mediated ferroptosis was investigated. Indeed, HTTQ94-mediated ferroptosis was largely abrogated by specific inhibitors of either FLAP or ALOX5 in both HT-22 (FIG. 5G) and SK-N-BE(2)C cells (FIG. 12D). Taken together, these data demonstrate that HTTQ94 activates ALOX5-mediated ferroptosis by interacting with FLAP and upregulating its stability.

### Example 6: Loss of ALOX5 Ameliorates the Pathological Phenotypes and Significantly Extends the Life Spans of these HD Mice

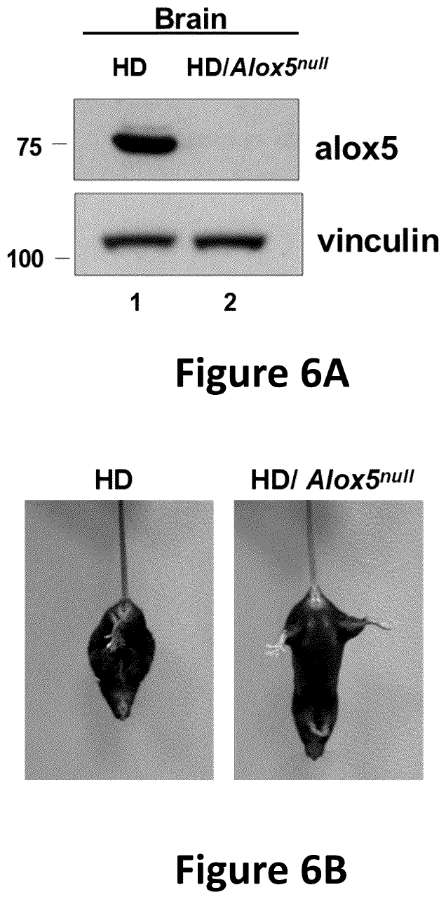

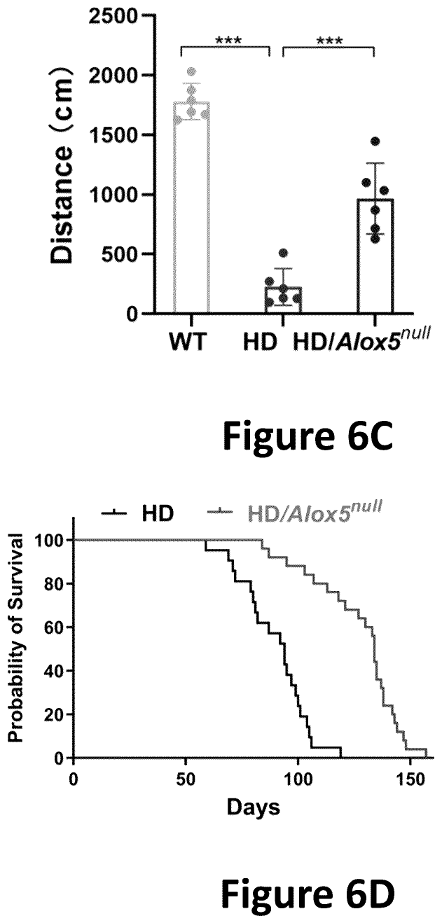

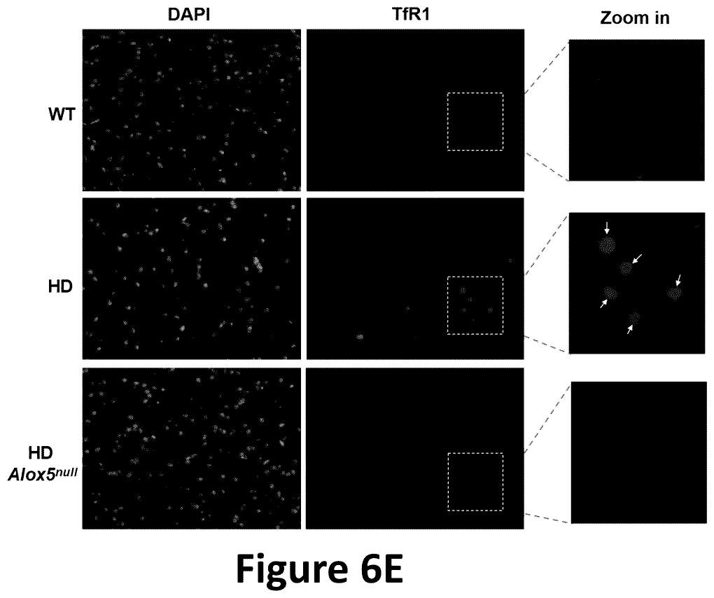

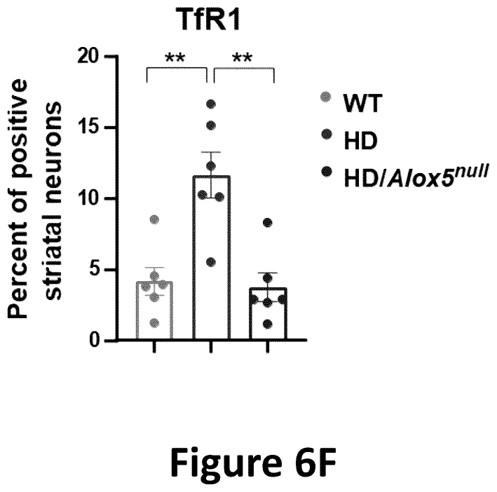

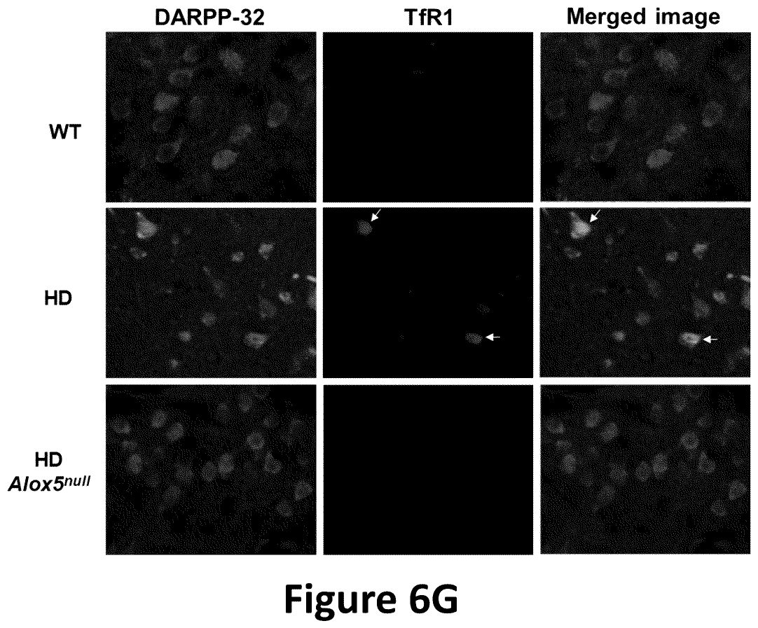

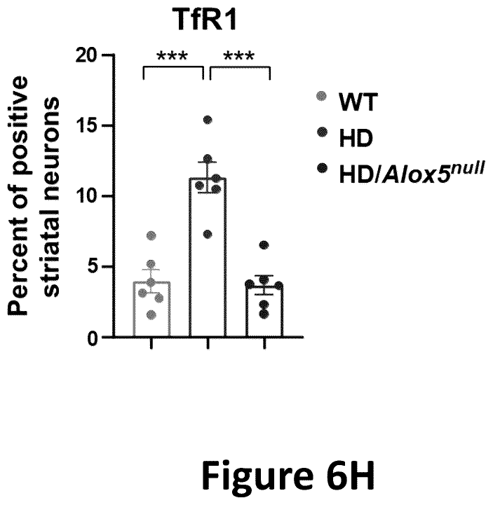

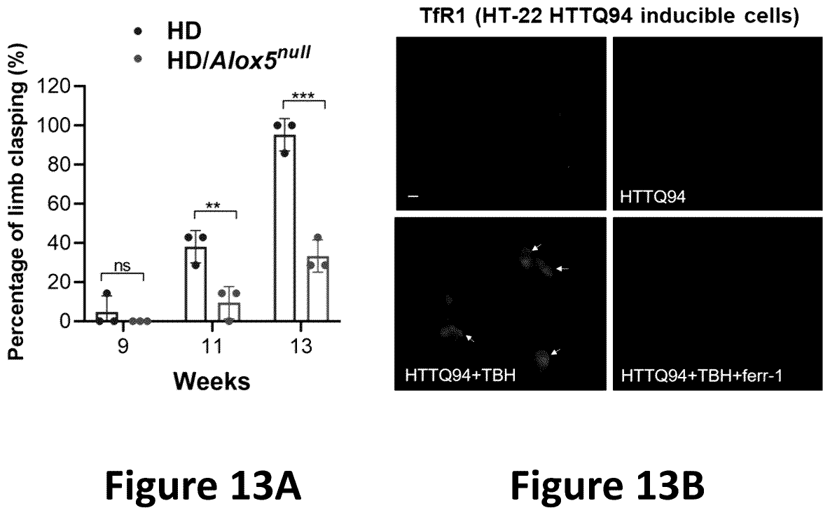

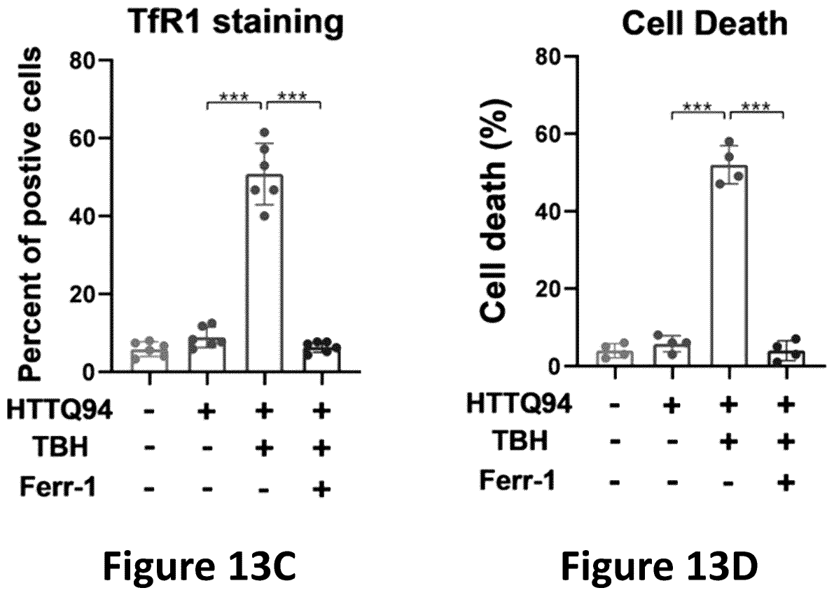

To examine the role of HTTQ94-mediated, ALOX5-dependent ferroptosis in contributing to the pathophysiological phenotypes in the HD mice, these HD mice (HD-N171-82Q) were crossed with Alox5-null mice to generate compound HD/Alox5null mutant mice. As shown in FIG. 6A, alox5 protein levels were undetectable in the brains of HD/Alox5null mutant mice. As expected, the HD-N171-82Q mice displayed a progressive neurological phenotype after 9 weeks old. For example, the clasping reflex, an abnormal posturing of the hind limb during the tail suspension, is a typical phenotype alteration characteristic of HD mice, that reflects the progression of brain damage (Schilling et al. 1999; Paul and Snyder 2019). The phenotypes of the HD/Alox5null mice were examined in comparison to the HD littermates. Strikingly, the levels of the HD mice showing clasping were significantly reduced in the HD/Alox5null mice at the same ages (FIG. 6B; FIG. 13A). Moreover, the travelled distance of these HD mice was significantly improved in the HD/Alox5null mice (FIG. 6C). More importantly, the HD/Alox5null mutant mice lived much longer than the HD control mice with the median survival increased to134 days, compared to 94 days for the HD mice (FIG. 6D).

To evaluate the role of ferroptosis in contributing to the pathological phenotypes of these mice, whether mHTT-mediated ferroptosis is indeed elevated in the HD mice and whether loss of ALOX5 is sufficient to abrogate the ferroptotic response in vivo were examined. Ferroptosis is a new type of programed cell death driven by the iron-dependent accumulation of lipid ROS. To further examine the levels of ferroptosis in the HD mice, a reliable way to specifically recognize ferroptotic cells in tissue sections to characterize the extent of ferroptosis in animal models had to be established. Notably, by screening ˜4,750 of the resulting monoclonal antibodies generated for their ability to selectively detect cells undergoing ferroptosis, one antibody, 3F3 ferroptotic membrane antibody (3F3-FMA) identified as the human transferrin receptor 1 protein (TfR1), is effective as a specific ferroptosis-staining reagent (Feng et al. 2020).

To validate whether this ferroptosis marker is effective at staining ferroptotic cells in the HD mice, whether the anti-TfR1 antibody staining is able to specifically recognize ferroptotic cells induced by HTTQ94 expression was first examined in mouse neuronal cells. To this end, the tet-on-HTTQ94 mouse hippocampal HT-22 neuronal inducible cell line was used. As shown in FIG. 13B, upon the induction of HTTQ94 expression in the presence of tert-butyl hydroperoxide (TBH), the levels of the anti-TfR1 antibody staining cells were dramatically increased. As expected, the anti-TfR1 antibody stained with greater intensity at the cell boundaries of those ferroptotic cells, and intracellular puncta also became brighter, and the levels of the staining were abolished in the presence of the ferroptosis inhibitor ferrostatin-1 (Ferr-1) (FIG. 13B). Moreover, the levels of the anti-TfR1 antibody staining were well correlated with the levels of ferroptosis cell death (FIG. 13C, D). These data indicate that the anti-TfR1 antibody staining is able to selectively stain ferroptotic cells induced by HTTQ94 expression. Next, the same assay was performed to examine the role of ALOX5 in modulating the ferroptosis levels in vivo. As shown in FIG. 6E, the levels of the bright staining with great intensity by anti-TfR1 antibody were very low in the wild-type mouse striatum; in contrast, the anti-TfR1 positive staining neurons were significantly increased in HD-N171-82Q mouse striatum. More importantly, ALOX5 deficiency completely abolished the levels of these anti-TfR1 positive staining neurons (FIG. 6E, F). Thus, by using the anti-TfR1 antibody staining, the fact that ALOX5 is crucial for ferroptotic cell death in the HD mice was validated. Since the medium spiny projection neuron cells constitutes ˜95% striatal neurons, it is very likely that the ferroptotic cells detected in the HD mice (HD-N171-82Q) are the medium spiny projection neurons. To this end, Dopamine- and CAMP-regulated phosphoprotein-32 kDa (DARPP-32) as a specific maker was used for striatal neurons (Naia et al. 2018). Indeed, all the striatal neuronal cells were stained by the anti-DARPP-32 antibody (Green color) (FIG. 6G); the ferroptotic neuronal cells from the HD brains were also brightly stained with the anti-TfR1 antibody (Red color). Notably, all the anti-TfR1 positive cells (Red color) were also stained with the anti-DARPP-32 antibody (Green color) (FIG. 6G). Moreover, the levels of ferroptotic striatal neurons with TfR1 positive staining were completely abolished in Alox5-null mice (FIG. 6G, H). Together, these data demonstrate that the levels of ferroptosis are indeed upregulated in the striatal neurons of the HD mice and that inactivation of the Alox5 gene effectively abrogates the ferroptosis activity in these HD mice.

Cell death induced by mHTT toxicity is a pathological hallmark in HD, characterized by significant neuronal loss in the striatum and cerebral cortex, followed by widespread neuronal loss in other brain regions (Thu et al. 2010; Nana et al. 2014; Bates et al. 2015). Previous studies have indicated that apoptosis is apparently not the major factor for the neuronal cell death in HD (Turmaine et al. 2000; Hickey and Chesselet 2003). Ferroptosis is a newly identified form of cell death that is morphologically, biochemically, and genetically distinct from other known forms of cell death (Dixon et al. 2012; Stockwell et al. 2017). Indeed, that expression of the mHTT fragment with the expanded 94 glutamine residues (HTTQ94) but not the wild type counterpart (HTTQ19) sensitizes neuronal cells to ferroptosis was found. Although numerous studies implicate ACSLA as a central factor in modulating ferroptotic responses induced by GPX4 inhibition, inactivation of the ACSL4-dependent ferroptosis did not have significant effect on pathophysiological phenotypes and the life span of the HD mice. Notably, HTTQ94 can also induce ferroptosis upon ROS-induced stress through a distinct regulatory pathway. Further, HTTQ94 mediated ferroptosis upon ROS stress is mediated by ALOX5. Moreover, HTTQ94 activates ALOX5 function through stabilizing its cofactor FLAP.

Signs and symptoms of Huntington's disease typically begin ages 30 to 50 and progress over the next 10 to 20 years (Bates et al. 2015), suggesting that besides mHTT toxic function, aging also plays an important role in neuronal death. The production of ROS is progressively increased in aging which is one of the key factors in cellular damage (Floyd and Hensley 2002; Tower 2015). Although the precise mechanism of mHTT-mediated ferroptosis in vivo needs further elucidation, based on the data described herein, it is very likely that mHTT-mediated ferroptotic responses are tightly regulated by the levels of ROS production in HD patients. Moreover, glutamate, the most common endogenous excitatory neurotransmitter in brain, plays a crucial role in neuronal tissue damage. Under normal physiological condition, glutamate regulates memory, learning, cognitive, emotional, endocrine and other visceral functions (Liu et al. 2015; Maher et al. 2020; Xie et al. 2022). Interestingly, the data described herein indicate that glutamate-induced ferroptosis is significantly enhanced by HTTQ94 expression. More importantly, the results show that ALOX5 is essential for these effects. Numerous studies indicate that glutamate-mediated cell death is involved in the pathogenesis of almost all neurological diseases, including Huntington's diseases (Coyle and Puttfarcken 1993; Caudle and Zhang 2009; Lewerenz and Maher 2015; McGrath et al. 2022). These data reveal a potential physiological pathway in modulating mHTT-induced ferroptosis during the pathological process of Huntington's diseases. Ferroptosis driven by lethal amounts of lipid peroxide is critical for ROS stress responses (Yang and Stockwell 2016). Lipid peroxidation has been observed in the brain of the HD mice (Lee et al. 2011; Skouta et al. 2014), indicating the involvement of ferroptosis in the progression of HD. The data described herein further showed that loss of ALOX5 expression abrogates ferroptosis in the striatal neurons of the HD mice. More importantly, the HD/Alox5″ull mutant mice survived significantly longer than HD mice with much improved pathological phenotypes. Phenotypes of the HD/Alox5″ull mutant mice can be further characterized and findings in the HD mouse model expressing full-length mHTT and other HD mouse models can be further validated using this model. Nevertheless, the findings described herein indicate that ALOX5-dependent ferroptosis pathway is crucial of mHTT-induced pathophysiological phenotypes of the HD mice and suggest that inhibition of the ALOX5 activity may be beneficial for treatment of Huntington's disease.

Although the role of the ACSL4-mediated ferroptosis in contributing to several types of neurodegenerative diseases has been well recognized, the data described herein implicates that a new ferroptosis pathway controlled by ALOX5 is critical for mHTT-mediated effects in HD mice. There are several additional mouse models of HD including the BAC transgenic or Knockin mice that express full length HTT. HdhQ111 mice accurately express 111-glutamine mutant huntingtin and exhibit early dominant abnormalities selective for medium spiny striatal neurons, including nuclear retention of full-length mutant huntingtin (Wheeler et al., 2000). A bacterial artificial chromosome (BAC)-mediated transgenic mouse model (BACHD) was developed expressing full-length-mHtt with 97 glutamine repeats under the control of endogenous htt regulatory machinery on the BAC (Gray et al., 2008). Both HD mice exhibit progressive motor deficits, neuronal synaptic dysfunction, and selective neuropathology, which includes significant cortical and striatal atrophy and striatal dark neuron degeneration. Whether loss of ALOX5 expression is able to effectively ameliorate pathological phenotypes and significantly extend the life spans of HD mice can be further studied. Moreover, mHTT-mediated ferroptosis is effectively blocked by Zileuton, a specific inhibitor of ALOX5. Interestingly, Zileuton is a FDA-approved drug that has been used for the treatment of asthma (Liu et al., 2015). The effect of this compound in the pathological phenotypes and the life spans of HD mice can be tested for repurposing this drug to therapeutic application in Huntington's disease.

### Methods

### Mammalian Cell Culture

The SK-N-BE(2)C neuroblastoma, H1299 and HEK293 cell lines were obtained from American Type Culture Collection (ATCC). The HT-22 Mouse Hippocampal Neuronal Cell Line was obtained from Sigma-Aldrich in 2018. All cells have been proven to be negative for mycoplasma contamination. No cell lines used in this work were listed in the ICLAC database. All cells were cultured in a 37° C. incubator with 5% CO2. All media were supplemented in DMEM with 10% FBS, 100 U/mL penicillin and 100 μg/mL streptomycin (all from Gibco). Stable cell lines derived from these cell lines and experimental treatments are described in method details.

### Plasmids

pTreTight-HTTQ94-GFP was a gift from Nico Dantuma (Addgene plasmid #23966; http://n2t.net/addgene:23966; RRID: Addgene_23966). HTTQ94-GFP was subcloned into pcDNA3.1/v5-His-Topo vector (Invitrogen). HTTQ94 sequence was amplified from pTreTight-HTTQ94-GFP. HTTQ19 (nHTT) sequence was amplified from the DNA isolated from Human cells. Both the HTTQ94 and HTTQ19 sequences were subcloned into pDONR221-vector and then were subcloned into pInducer-vector (Gateway LR kit, Thermo) to generate pInducer-HTTQ19 and pInducer-HTTQ94 respectively. V5-ALOX5 was previously described (Chu et al., 2019). pDONR221-FLAP was obtained from Harvard Medical School PlasmID (HsCD00043730). To generate HA-FLAP and Flag-FLAP, full length FLAP was amplified using forward primer with HA sequence and Flag sequence respectively, and then was subcloned into pcDNA3.1/v5-His-Topo vector (Invitrogen). cDNA of AIFM2 (FSP1), GCH1 and DHODH was amplified using forward primer with Flag sequence and cloned into pcDNA3.1/v5-His-Topo vector (Invitrogen).

### Cell Lines Construction

To generate the HTTQ94 and HTTQ19 fragments Tet-on stable cell lines, pInducer-HTTQ94 and pInducer-HTTQ19 were transfected into H1299 cells. To generate the HTTQ94 Tet-on stable neuronal cell lines, pInducer-HTTQ94 was transfected into HT-22 and SK-N-BE(2)C cells. The transfected cells were selected and maintenanced with 500 μg/mL G418 (Sigma) in DMEM medium containing 10% FBS. Single clones were selected and screened by western blot. ACSL4 and ALOX5 CRISPR-cas9-knockout cells were generated by transfecting ACSL4 and ALOX5 double nickase plasmid (ACSL4, sc-401649-NIC; ALOX5, sc-401239-NIC; Santa Cruz) into the HTTQ94 Tet-on SK-N-BE(2)C cells. ACSL4/GPX4 double-knockout cells were generated by transfecting ACSL4 and GPX4 double nickase plasmid (GPX4, sc-401558-NIC; Santa Cruz) simutaneously into the HTTQ94 Tet-on SK-N-BE(2)C cells. Forty-eight hours later, CRISPR efficiency was determined by Western blot analysis, and then pool cells were seeded in 10-cm dish to grow clones at a density of 100-200 cells/dish. One or two weeks later, moloclones were picked and seeded into 12-well plates, followed by identification by Western blot analysis.

### Western Blotting and Antibodies

Protein extracts were analyzed by Western Blotting according to standard protocols using primary antibodies specific for ALOX5 (3289S; Cell signaling; 1:500 dilution), ACSL4 antibody (A5) (sc-271800; Santa Cruz; 1:1000 dilution), HA (11867423001; Sigma; 1:1000 dilution), Flag (F-3040;Sigma; 1:1000 dilution), FLAP (ab85227; Abcam; 1:1000 dilution), β-actin (Ab8227; Abcam; 1:2000 dilution) and vinculin (V9264; Sigma-Aldrich; 1:5000 dilution). HRP-conjugated anti-mouse (1031-05; SouthernBiotech) and anti-rabbit secondary antibody (4050-05; SouthernBiotech) and anti-rat secondary antibody (3051-05; Southern Biotech) were used.

### RNA Interference

Cells were plated at 20˜30% density one day prior to siRNAs transfection. Knockdown of ALOX family proteins and FLAP was performed by transfection of HTTQ94 Tet-on SK-N-BE(2)C cells with 60 μM siRNA duplex oligoset (ON-TARGET plus SMARTpool: ALOXE3: L-009022-00-0005; ALOX12B: L-009025-00-0005; ALOX12: L-004558-00-0005; ALOX5: L-004530-00-0005; ALOX15B: L-009026-00-0005; ALOX15: L-003808-00-0005; FLAP: L-010166-01-0005; Horizon Discovery) with Lipofectamine 3000 (Invitrogen) according to the manufacturer's protocol. HTTQ94 Tet-on HT-22 cells were transfected with 60 μM mouse ALOX5 siRNA duplex oligo set (ON-TARGET plus SMARTpool, L-065695-01-0005, Horizon Discovery). HTTQ94 Tet-on SK-N-BE(2)C cells were transfected with 40 μM AIFM2/FSP1, GCH1 and DHODH siRNAs pool (Invitrogen, Shanghai, China). Cells were collected at 48˜72 h after transfection and subjected to functional assays. The siRNA target sequences were follows:

AIFM2/FSP1,
siRNA-1,
5′- CAACAUCGUCAACUCUGUGAA -3′
and
siRNA-2,
5′- GAUUCUCUGCACCGGCAUCAA -3′;
GCH1,
siRNA-1,
5′- GCGAGGAUUGUAGAAAUCUAU -3′
and
siRNA-2,
5′- GCAACACACAUGUGUAUGGUA -3′;
DHODH,
siRNA-1,
5′- GUGAGAGUUCUGGGCCAUAAA -3′
and
siRNA-2,
5′- CGAUGGGCUGAUUGUUACGAA -3′.

RNA Extraction and qRT-PCR.

Total RNA was extracted using TRIzol (Thermo Fisher Scientific, Cat #15596018) according to the manufacturer's protocol. cDNA was generated using SuperScript IV VILO Master Mix (Thermo Fisher Scientific, Cat #11756500). Quantitative PCR was done using a 7500 Fast Real-Time PCR System (Applied Biosystems) with standard protocol. Reactions were done in triplicate. The following primers were used: human ALOX family and mouse CHAC1 described previously (Chu et al., 2019); human FLAP forward 5′-CTTGCCTTTGAGCGGGTCTA-3′, reverse 5′-CATCAGTCCAGCAAACGCAG-3′; mouse ALOX5 forward 5′-ATCGAGTTCCCATGTTACCGC-3′, reverse 5′-AATTTGGTCATCTCGGGCCA-3′; human AIFM2/FSP1 forward 5′-AGACAGGGTTCGCCAAAAAGA-3′, reverse 5′-CAGGTCTATCCCCACTACTAGC-3′; human GCH1 forward 5′-ACGAGCTGAACCTCCCTAAC-3′, reverse 5′-GAACCAAGTGATGCTCACACA-3′; human DHODH forward 5′-GTTCTGGGCCATAAATTCCGA-3′, reverse 5′-TCTGGGTCTAGGGTTTCCTTC-3′.

### Drugs and Cell Death Inhibitors

All drugs (except for drugs below) were ordered from Sigma-Aldrich. Ferrostatin-1 was from Xcess Biosciences; MK886 were from MedChemExpress (MCE); For ROS generation, tert-butyl hydroxide solution (TBH) was used at different doses depending on the experiment; see respective figure legends. Erastin (ferroptosis inducer), Rosiglitazone (ACSL4 inhibitor), Troglitazone (ACSLA inhibitor), Zileuton (ALOX5 inhibitor), MK886 (FLAP inhibitor), Ferrostatin-1 (ferroptosis inhibitor), DFO (ferroptosis inhibitor), Liproxstatin-1 (ferroptosis inhibitor), 3-methylademine (autophagy inhibitor), Z-VADFMK (apoptosis inhibitor) and necrostatin-1(necroptosis inhibitor) were used at different doses depending on the experiment; see related figure legends.

### Cell Death Assay

Cells were treated with ferroptosis inducer TBH or erastin or glutamate combined with other drugs or conditions. At indicated time points, cells were trypsinized and stained with trypan blue followed by counting with a hemocytometer using the cell number counter (Life technologies countess II). Living cells and dead cells were all counted, and cells stained blue were considered as dead cells. Quantification of cell death was further confirmed using ToxiLight Non-destructive Cytotoxicity BioAssay Kit (LT07-117; Lonza). Data were collected using GloMax® Explorer Multimode Microplate Reader (Promega).

### Lipid ROS Assay Using Flow Cytometer.

Cells were incubated with DMEM containing 5 μM of BODIPY™ 581/591 C11 (D3861; Thermo Fisher Scientific) at the concentration of 5 μM for 25-30 min at 37° C. in serum-free medium. Cells were then harvested and washed twice with PBS followed by re-suspending in 500 μl of PBS. Lipid ROS levels were analyzed using a Becton Dickinson FACS Calibur machine through the FL1 channel, and the data were analyzed using CellQuest software. 10000 cells were collected and analyzed for each sample.

### Mice.

The Alox5 knockout mice and Huntington transgenic mouse (HD-N171-82Q) were purchased from the Jackson Laboratory (stock number 004155 and 003627, respectively). The Acs14 knockout mice were described previously (Chu et al., 2019) . . . . All experimental protocols using mice were approved by the Institutional Animal Care and Use Committee (IACUC) of Columbia University.

### Immunofluorescence Staining

For cell line: HT-22 HTTQ94 inducible cells were fixed with 2% paraformaldehyde at RT for 20 min, and then incubated with blocking buffer (10% donkey serum, 0.3% Triton X-100 in PBS), followed by incubation with TfR1 antibody (1: 250, Thermo Fisher Scientific, Cat #13-6800) for 1 h, and then incubation with Alexa 568 conjugated secondary antibody (1:250, Thermo Fisher Scientific, Cat #A11004) for additional 1 h.

For mouse brain slides: brains paraffin slides were dewaxed by xylene, and antigens were retrieved with PH6.0 citrate buffer using pressure cooker. Slides were blocked with 5% horse serum for 30 min, and then incubated with primary antibody (TfR1: 1:1000, Thermo Fisher Scientific, Cat #13-6800; DARPP-32: 1:200, Cell Signaling, Cat #2306) for 1-1.5 h, followed by incubation with biotinylated secondary antibody (1: 200, Vector Laboratories, Cat #BA-2000; Cat #BA-1000) for 30 min, and then incubation with streptavidin conjugated Alexa 594/488 dye (1:1000, Thermo Fisher Scientific, Cat #S32356; Cat #S32354) or another 30 min. In double staining experiment, Alexa 488 conjugated secondary antibody (Thermo Fisher Scientific, Cat #A11008) was also used for detecting DARPP-32.

### Behavioral Tests

Both female and male mice were included for behavioral tests. There was no significant effect on gender for the behavioral tests. Motor activity was measured at 13 weeks of age in an open field. Mice were allowed to habituate to the experimental environment for at least one hour before assessment. Mice were placed in the center for 10 minutes, during which the distance travelled was recorded for the last 5 minutes by a digital camera coupled to the Smart Junior system. The distance travelled within each group was averaged separately. As for the clasping test, each mouse was suspended upside down by its tail for 15 seconds. They were administered three trials a week at 9, 11 and 13 weeks of age. The weekly percentage of limb clasping within each group was averaged separately. The weight of each mouse was recorded weekly.

### Statistical Analysis.

Statistical analysis was carried out using GraphPad Prism 9 software. Results are presents as the mean±s.d. or ±s.e.m. Statistical significance of the mice Kaplan-Meier survival curves was determined by log-rank Mantel-Cox test.

### REFERENCES

Badgley M A, Kremer D M, Maurer H C, DelGiorno K E, Lee H J, Purohit V, Sagalovskiy I R, Ma A, Kapilian J, Firl C E M et al. 2020. Cysteine depletion induces pancreatic tumor ferroptosis in mice. Science 368: 85-89.
Bates G. 2003. Huntingtin aggregation and toxicity in Huntington's disease. The Lancet 361: 1642-1644.
Bates G P, Dorsey R, Gusella J F, Hayden M R, Kay C, Leavitt B R, Nance M, Ross C A, Scahill R I, Wetzel R et al. 2015. Huntington disease. Nat Rev Dis Primers 1: 15005.
Bersuker K, Hendricks J M, Li Z, Magtanong L, Ford B, Tang P H, Roberts M A, Tong B, Maimone T J, Zoncu R et al. 2019. The CoQ oxidoreductase FSP1 acts parallel to GPX4 to inhibit ferroptosis. Nature 575: 688-692.
Caudle W M, Zhang J. 2009. Glutamate, excitotoxicity, and programmed cell death in Parkinson disease. Exp. Neurol 220: 230-233.
Chu B, Kon N, Chen D, Li T, Liu T, Jiang L, Song S, Tavana O and Gu W. 2019. ALOX12 is required for p53-mediated tumour suppression through a distinct ferroptosis pathway. Nat cell boil 21: 579.
Coyle J T, Puttfarcken P. 1993. Oxidative stress, glutamate, and neurodegenerative disorders. Science 262: 689-695.
Dixon S J, Lemberg K M, Lamprecht M R, Skouta R, Zaitsev E M, Gleason C E, Patel D N, Bauer A J, Cantley A M, Yang W S et al. 2012. Ferroptosis: an iron-dependent form of nonapoptotic cell death. Cell 149: 1060-1072.
Doll S, Proneth B, Tyurina Y Y, Panzilius E, Kobayashi S, Ingold I, Irmler M, Beckers J, Aichler M, Walch A et al. 2017. ACSL4 dictates ferroptosis sensitivity by shaping cellular lipid composition. Nat Chem Biol 13: 91.
Doll S, Freitas F P, Shah R, Aldrovandi M, da Silva M C, Ingold I, Goya Grocin A, Xavier da Silva T N, Panzilius E, Scheel C H et al. 2019. FSP1 is a glutathione-independent ferroptosis suppressor. Nature 575: 693-698.
Feng H, Schorpp K, Jin J, Yozwiak C E, Hoffstrom B G, Decke A M, . . . & Stockwell B R. 2020. Transferrin receptor is a specific ferroptosis marker. Cell reports 30: 3411-3423.
Floyd R A & Hensley K. 2002. Oxidative stress in brain aging: implications for therapeutics of neurodegenerative diseases. Neurobiol Aging 23: 795-807.
Graham R K, Deng Y, Slow E J, Haigh B, Bissada N, Lu G, Pearson J, Shehadeh J, Bertram L, Murphy Z et al. 2006. Cleavage at the caspase-6 site is required for neuronal dysfunction and degeneration due to mutant huntingtin. Cell 125: 1179-1191.
Gray M, Shirasaki D I, Cepeda C, André V M, Wilburn B, Lu X H, Tao J, Yamazaki I, Li S H, Sun Y E, Li X J, Levine M S, Yang X W. 1998 Full-length human mutant huntingtin with a stable polyglutamine repeat can elicit progressive and selective neuropathogenesis in BACHD mice. J Neurosci. 28: 6182-95.
Hickey M A & Chesselet M F. 2003. Apoptosis in Huntington's disease. Prog Neuropsychopharmacol Biol Psychiatry 27: 255-265.
Jo A, Bac J H, Yoon Y J, Chung T H, Lec E W, Kim Y H, Joh H M & Chung J W. 2022. Plasma-activated medium induces ferroptosis by depleting FSP1 in human lung cancer cells. Cell death & disease 13: 212.
Kagan V E, Mao G, Qu F, Angeli J P, Doll S, Croix C S, Dar H H, Liu B, Tyurin V A, Ritov V B et al. 2017. Oxidized arachidonic and adrenic PEs navigate cells to ferroptosis. Nat Chem Biol 13: 81-90.
Klepac N, Relja M, Klepac R, Hećimović S, Babić T, Trkulja V J. 2007. Oxidative stress parameters in plasma of Huntington's disease patients, asymptomatic Huntington's disease gene carriers and healthy subjects: a cross-sectional study. J Neurol 254: 1676-1683
Kraft V, Bezjian C T, Pfeiffer S, Ringelstetter L, Müller C, Zandkarimi F, Merl-Pham J, Bao X, Anastasov N, Kössl J et al. 2020. GTP Cyclohydrolase 1/Tetrahydrobiopterin Counteract Ferroptosis through Lipid Remodeling. ACS central science 6: 41-53.
Kumar P, Kalonia H, Kumar A. 2010. Nitric oxide mechanism in the protective effect of antidepressants against 3-nitropropionic acid-induced cognitive deficit, glutathione and mitochondrial alterations in animal model of Huntington's disease. Behav Pharmacol 21: 217-230.
Lee J, Kosaras B, Del Signore S J, Cormier K, McKee A, Ratan R R, Kowall N W and Ryu H. 2011. Modulation of lipid peroxidation and mitochondrial function improves neuropathology in Huntington's disease mice. Acta Neuropathol 121: 487-498.
Lewerenz J & Maher P. 2015. Chronic Glutamate Toxicity in Neurodegenerative Diseases-What is the Evidence?. Front Neurosci 9: 469.
Liu Y, Wang W, Li Y, Xiao Y, Cheng J, Jia J. 2015 The 5-Lipoxygenase Inhibitor Zileuton Confers Neuroprotection against Glutamate Oxidative Damage by Inhibiting Ferroptosis. Biol Pharm Bull 38: 1234-1239.
Lunkes A, Lindenberg K S, Ben-Haïem L, Weber C, Devys D, Landwehrmeyer G B, Mandel J L and Trottier Y. 2002. Proteases acting on mutant huntingtin generate cleaved products that differentially build up cytoplasmic and nuclear inclusions. Mol Cell 10: 259-269.
MacDonald M E, Ambrose C M, Duyao M P, Myers R H, Lin C, Srinidhi L, Barnes G, Taylor S A, James M, Groot N et al. 1993. A novel gene containing a trinucleotide repeat that is expanded and unstable on Huntington's disease chromosomes. Cell 72: 971-983.
Maher P, Currais A, Schubert D. 2020. Using the Oxytosis/Ferroptosis Pathway to Understand and Treat Age-Associated Neurodegenerative Diseases. Cell Chem Biol 27: 1456-1471.
Mao C, Liu X, Zhang Y, Lei G, Yan Y, Lee H, Koppula P, Wu S, Zhuang L, Fang B et al. 2021. DHODH-mediated ferroptosis defence is a targetable vulnerability in cancer. Nature 593: 586-590.
Martindale D, Hackam A, Wieczorek A, Ellerby L, Wellington C, Mccutcheon K, Singaraja R, Kazemi-Esfarjani P, Devon R, Kim S U et al. 1998. Length of huntingtin and its polyglutamine tract influences localization and frequency of intracellular aggregates. Nat Genet 18: 150.
Mashima R, Okuyama T. 2015. The role of lipoxygenases in pathophysiology; new insights and future perspectives. Redox Biol 6: 297-310.
McGrath T, Baskerville R, Rogero M & Castell L. 2022. Emerging Evidence for the Widespread Role of Glutamatergic Dysfunction in Neuropsychiatric Diseases. Nutrients 14: 917.
Nana A L, Kim E H, Thu D C, Oorschot D E, Tippett L J, Hogg V M, Synek B J, Roxburgh R, Waldvogel H J, Faull R L. 2014. Widespread heterogeneous neuronal loss across the cerebral cortex in Huntington's disease. J Huntingtons Dis 3: 45-64.
Naia L & Rego A C. 2018. Isolation and Maintenance of Murine Embryonic Striatal Neurons. Bio-protocol 8(8): e2823
Paul B D & Snyder S H. 2019. Impaired redox signaling in Huntington's disease: therapeutic implications. Front Mol Neurosci 12: 68.
Peters-Golden M, Brock T G. 2003. 5-lipoxygenase and FLAP. Prostaglandins Leukot Essent Fatty Acids 69: 99-109.
Pergola C, Gerstmeier J, Mönch B, çalιşkan B, Luderer S, Weinigel C, Barz D, Maczewsky J, Pace S, Rossi A et al. 2014. The novel benzimidazole derivative BRP-7 inhibits leukotriene biosynthesis in vitro and in vivo by targeting 5-lipoxygenase-activating protein (FLAP). Br J Pharmacol 171: 3051-3064.
Savonenko A V, Klevytska A, Morton J L, Tucker S M, Poirier M, Gale A, Chan N, Gonzales V, Slunt H H et al. 2004. Nuclear-targeting of mutant huntingtin fragments produces Huntington's disease-like phenotypes in transgenic mice. Hum Mol Genet 13: 1599-1610.
Schilling G, Becher M W, Sharp A H, Jinnah H A, Duan K, Kotzuk J A, Slunt H H, Ratovitski T, Cooper J K, Jenkins N A et al. 1999. Intranuclear inclusions and neuritic aggregates in transgenic mice expressing a mutant N-terminal fragment of huntingtin. Hum Mol Genet 8: 397-407.
Stockwell B R, Angeli J P F, Bayir H, Bush A I, Conrad M, Dixon S J, Fulda S, Gascón S, Hatzios S K, Kagan V E et al. 2017. Ferroptosis: a regulated cell death nexus linking metabolism, redox biology, and disease. Cell 171: 273-285.
Stockwell B R, Jiang X & Gu W. 2020. Emerging mechanisms and disease relevance of ferroptosis. Trends Cell Biol 30: 478-490.
Skouta R, Dixon S J, Wang J, Dunn D E, Orman M, Shimada K, Rosenberg P A, Lo D C, Weinberg J M, Linkermann A et al. 2014. Ferrostatins inhibit oxidative lipid damage and cell death in diverse disease models. J Am Chem Soc 136: 4551-4556.
Thu D C, Oorschot D E, Tippett L J, Nana A L, Hogg V M, Synek B J, Luthi-Carter R, Waldvogel H J, Faull R L. 2010. Cell loss in the motor and cingulate cortex correlates with symptomatology in Huntington's disease. Brain 133: 1094-1110.
Tower J. Programmed cell death in aging. 2015. Ageing Res Rev 23: 90-100.
Turmaine M, Raza A, Mahal A, Mangiarini L, Bates G P & Davies S W. 2000. Nonapoptotic neurodegeneration in a transgenic mouse model of Huntington's disease. Proc Natl Acad Sci 97: 8093-8097.
Wheeler, V. C., White, J. K., Gutekunst, C. A., Vrbanac, V., Weaver, M., Li, X. J., Li, S. H., Yi, H., Vonsattel, J. P., Gusella, J. F. et al. (2000) Long glutamine tracts cause nuclear localization of a novel form of huntingtin in medium spiny striatal neurons in HdhQ92 and HdhQ111 knock-in mice. Hum. Mol. Genet., 9: 503-513.
Xie Z, Xu M, Xie J, Liu T, Xu X, Gao W, Li Z, Bai X, Liu X. 2022. Inhibition of Ferroptosis Attenuates Glutamate Excitotoxicity and Nuclear Autophagy In A CLP Septic Mouse Model. Shock 57: 694-702.
Yablonska S, Ganesan V, Ferrando L M, Kim J, Pyzel A, Baranova O V, Khattar N K, Larkin T M, Baranov S V, Chen N et al. 2019. Mutant huntingtin disrupts mitochondrial proteostasis by interacting with TIM23. Proc Natl Acad Sci 116: 16593-16602.
Yang W S & Stockwell B R. 2016. Ferroptosis: death by lipid peroxidation. Trends Cell Biol 26: 165-176.

What is claimed is:

1. A method of treating or preventing Huntington's disease in a subject in need thereof, comprising administering to said subject a composition that targets an acyl-CoA synthetase long-chain family member 4 (ACSL4)-independent ferroptosis pathway.

2. A method of treating or preventing Huntington's disease in a subject in need thereof, comprising:
(i) identifying the subject as expressing mutant huntingtin (mHTT), and
(ii) administering to said subject a composition that targets an acyl-CoA synthetase long-chain family member 4 (ACSLA)-independent ferroptosis pathway.

3. A method of treating or preventing Huntington's disease in a subject in need thereof, comprising:
(i) identifying the subject as having an increased expression level of arachidonate 5-lipoxygenase (ALOX5), and
(ii) administering to said subject a composition that targets an acyl-CoA synthetase long-chain family member 4 (ACSL4)-independent ferroptosis pathway.

4. A method of treating or preventing Huntington's disease in a subject in need thereof, comprising:
(i) identifying the subject as having an increased expression level of 5-lipoxygenase-activating protein (FLAP), and
(ii) administering to said subject a composition that targets an acyl-CoA synthetase long-chain family member 4 (ACSLA)-independent ferroptosis pathway.

5. The method of claim 1, wherein the subject expresses mHTT, an increased level of ALOX5, an increased level of FLAP, or a combination thereof.

6. The methods of claim 2 wherein expression of mHTT is determined from a sample from the subject.

7. The method of claims 3-4, wherein expression level is determined from a sample from the subject.

8. The method of claim 6 or 7, wherein expression or expression level is a protein level, a mRNA expression level, or combination thereof.

9. The method of claim 2, wherein the subject further expresses an increased level of ALOX5, an increased level of FLAP, or a combination thereof.

10. The method of claim 3, wherein the subject further expresses mHTT, an increased level of FLAP, or a combination thereof.

11. The method of claim 4, wherein the subject further expresses mHTT, an increased level of ALOX5, or a combination thereof.

12. The method of any one of claims 1-11, wherein the composition reduces ALOX5 expression in the subject compared to ALOX5 expression in a subject suffering from Huntington's disease or compared to ALOX5 expression in the subject before administration of the composition.

13. The method of any one of claims 1-12, wherein the composition reduces FLAP expression in the subject compared to FLAP expression in a subject suffering from Huntington's disease or compared to FLAP expression in the subject before administration of the composition.

14. The method of any one of claims 1-13, wherein the composition inhibits or reduces ALOX5 expression in the subject.

15. The method of any one of claims 1-13, wherein the composition inhibits or reduces FLAP expression in the subject.

16. The method of any one of claims 1-15, wherein the composition comprises Zileuton.

17. The method of any one of claims 1-15, wherein the composition comprises MK.886.

18. The method of any one of claims 1-15, wherein the composition comprises docebenone (AA 861).

19. The method of any one of claims 1-15, wherein the composition comprises boswellic acids.

20. The method of any one of claims 1-15, wherein the composition comprises atreleuton (ABT-761 or VIA-2291).

21. The method of any one of claims 1-15, wherein the composition comprises setileuton (1,3,4-oxadiazole MK-0633

22. The method of any one of claims 1-15, wherein the composition comprises PF-4191834 or CJ-13610.

23. The method of any one of claims 1-15, wherein the composition comprises Flavocoxid.

24. The method of any one of claims 1-15, wherein the composition comprises an ALOX5 small interfering ribonucleic acid (siALOX5).

25. The method of any one of claims 1-15, wherein the composition comprises a FLAP small interfering ribonucleic acid (siFLAP).

26. The method of claims 1-15, wherein the composition comprises an ALOX5 short-hairpin ribonucleic acid (shALOX5).

27. The method of claims 1-15, wherein the composition comprises a FLAP short-hairpin ribonucleic acid (shFLAP).

28. The method of any one of claims 1-15, wherein the composition comprises a guide RNA or a single-molecule guide RNA comprising a spacer sequence that is complementary to a portion of a nucleic acid sequence encoding ALOX5.

29. The method of any one of claims 1-15, wherein the composition comprises a guide RNA or a single-molecule guide RNA comprising a spacer sequence that is complementary to a portion of a nucleic acid sequence encoding FLAP.

30. The method of claim 28 or 29, wherein the guide RNA or the single molecule guide RNA is pre-complexed with a DNA endonuclease.

31. The method of claim 30, wherein the DNA endonuclease is a Cas9 or dCas9 endonuclease.

32. The method of any one of claims 24-29, wherein the composition comprises a vector.

33. The method of any one of claims 26-29, wherein the composition comprises a viral vector, wherein the viral vector encapsulates a nucleic acid encoding the shALOX5, a nucleic acid encoding the shFLAP, a nucleic acid encoding the guide ribonucleic acid or a single molecule guide RNA comprising a spacer sequence that is complementary to a portion of a nucleic acid sequence encoding ALOX5, or a nucleic acid encoding the guide ribonucleic acid or a single molecule guide RNA comprising a spacer sequence that is complementary to a portion of a nucleic acid sequence encoding FLAP.

34. The method of claim 33, wherein the viral vector is an adeno-associated vector (AAV).

35. The method of any one of claims 1-34, wherein the subject is a mammal.

36. The method of claim 35, wherein the mammal is a human.

37. The method of claim 36, wherein the human subject has an increased expression level of ALOX5 compared to a human subject not suffering from Huntington's disease.

38. The method of claim 36, wherein the human subject has an increased expression level of FLAP compared to a human subject not suffering from Huntington's disease.

39. The method of claims 1-38, wherein the composition is delivered systemically.

40. A composition for treating or preventing Huntington's disease, comprising a composition targeting the ACSLA-independent ferroptosis pathway.

41. The composition of claim 40, wherein the composition reduces arachidonate 5-lipoxygenase (ALOX5) expression in a subject in need thereof.

42. The composition of claim 40 or 41, wherein the composition reduces 5-lipoxygenase-activating protein (FLAP) expression in a subject in need thereof.

43. The composition of any one of claims 40-42, wherein the composition comprises Zileuton.

44. The composition of any one of claims 40-42, wherein the composition comprises MK.886.

45. The composition of any one of claims 40-42, wherein the composition comprises docebenone (AA 861).

46. The composition of any one of claims 40-42, wherein the composition comprises boswellic acids.

47. The composition of any one of claims 40-42, wherein the composition comprises atreleuton (ABT-761 or VIA-2291).

48. The composition of any one of claims 40-42, wherein the composition comprises setileuton (1,3,4-oxadiazole MK-0633).

49. The composition of any one of claims 40-42, wherein the composition comprises PF-4191834 or CJ-13610.

50. The composition of any one of claims 40-42, wherein the composition comprises flavocoxid.

51. The composition of any one of claims 40-42, wherein the composition comprises an ALOX5 small interfering ribonucleic acid (siALOX5).

52. The composition of any one of claims 40-42, wherein the composition comprises a FLAP small interfering ribonucleic acid (siFLAP).

53. The composition of any one of claims 40-42, wherein the composition comprises an ALOX5 short-hairpin ribonucleic acid (shALOX5).

54. The composition of any one of claims 40-42, wherein the composition comprises a FLAP short-hairpin ribonucleic acid (shFLAP).

55. The composition of any one of claims 40-42, wherein the composition comprises a guide ribonucleic acid or a single molecule guide RNA comprising a spacer sequence that is complementary to a portion of a nucleic acid sequence encoding ALOX5.

56. The composition of any one of claims 40-42, wherein the composition comprises a guide ribonucleic acid or a single molecule guide RNA comprising a spacer sequence that is complementary to a portion of a nucleic acid sequence encoding FLAP.

57. The composition of any one of claim 55 or 56, wherein the guide RNA or the single molecule guide RNA is pre-complexed with a DNA endonuclease.

58. The composition of claim 57, wherein the DNA endonuclease is a Cas9 or dCas9 endonuclease.

59. The composition of any one of claims 53-58, wherein the composition further comprises a viral vector, wherein the viral vector encapsulates a nucleic acid encoding the shALOX5, a nucleic acid encoding the shFLAP, a nucleic acid encoding the guide ribonucleic acid or a single molecule guide RNA comprising a spacer sequence that is complementary to a portion of a nucleic acid sequence encoding ALOX5, or a nucleic acid encoding the guide ribonucleic acid or a single molecule guide RNA comprising a spacer sequence that is complementary to a portion of a nucleic acid sequence encoding FLAP.

60. The composition of claim 59, wherein the viral vector is an AAV vector.

61. A small interfering ribonucleic acid (siRNA) targeting the acyl-CoA synthetase long-chain family member 4 (ACSL4)-independent ferroptosis pathway.

62. The siRNA of claim 61, wherein the siRNA comprises an ALOX5 small interfering ribonucleic acid (siALOX5).

63. The siRNA of claim 61, wherein the siRNA comprises a FLAP small interfering ribonucleic acid (siFLAP).

64. A vector comprising the siRNA of any one of claims 61-63.

65. A nucleic acid comprising a sequence encoding a short hairpin ribonucleic acid (shRNA) targeting the acyl-CoA synthetase long-chain family member 4 (ACSLA)-independent ferroptosis pathway.

66. The nucleic acid of claim 65, wherein the shRNA is an ALOX5 short-hairpin ribonucleic acid (shALOX5).

67. The nucleic acid of claim 65, wherein the shRNA is a FLAP short-hairpin ribonucleic acid (shFLAP).

68. A vector comprising the nucleic acid of any one of claims 65-67.

69. A viral vector comprising the nucleic acid of any one of claims 65-67.

70. The viral vector of claim 69, wherein the viral vector is an AAV vector.

71. A method of diagnosing Huntington's disease in a subject comprising:
(i) determining the level of arachidonate 5-lipoxygenase (ALOX5) in the subject; and
(ii) diagnosing the subject with Huntington's disease if the level of ALOX5 expression in the subject is increased as compared to a subject not suffering from Huntington's disease.

72. A method of diagnosing Huntington's disease in a subject comprising:
(i) determining the level of 5-lipoxygenase-activating protein (FLAP) in the subject; and
(ii) diagnosing the subject with Huntington's disease if the level of FLAP expression in the subject is increased as compared to a subject not suffering from Huntington's disease.
